(19) United States
(12) Patent Application Publication
(10) Pub. No.: US 2012/0035265 A1
(43) Pub. Date: Feb. 9, 2012

(54) COMPOSITIONS, METHODS, AND DEVICES FOR THE TREATMENT OF DYSMENORRHEA

(75) Inventors: Kazuko MATSUDA, Beverly Hills, CA (US); Yuichi Iwaki, Palos Verdes Estates, CA (US); Maria Feldman, San Diego, CA (US); Kale Ruby, Cardiff by the Sea, CA (US)

(73) Assignee: MediciNova, Inc.

(21) Appl. No.: 13/042,214

(22) Filed: Mar. 7, 2011

Related U.S. Application Data
(60) Provisional application No. 61/311,676, filed on Mar. 8, 2010.

(51) Int. Cl. A61K 31/196; A61P 15/00; A61P 9/00; A61P 23/02; A61P 21/00; A61K 31/165; A61P 29/00

(52) U.S. Cl. 514/567; 514/619

(57) ABSTRACT

A method for a prevention and/or treatment of dysmenorrhea or amelioration of a symptom thereof in a subject, comprising administering to said subject an effective amount of a compound of formula I:

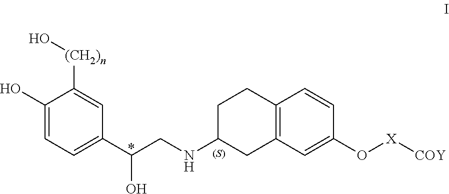

### CROSS-REFERENCE TO RELATED PATENT APPLICATIONS

This application claims priority from Provisional Application U.S. Application 61/311,676, filed Mar. 8, 2010, incorporated herein by reference in its entirety.

### BACKGROUND OF THE INVENTION

Uterine contractility disorders are significant health problems. Dysmenorrhea, painful uterine contractions or cramping during the menstrual period, affects gonadal women. The etiology of uterine contractility disorders are largely unknown and effective therapy to inhibit uterine contractility and prevent the symptoms associated with these diseases are unknown.

Dysmenorrhea, which may be primary or secondary, is the occurrence of painful uterine cramps during menstruation. In secondary dysmenorrhea, there is a visible pelvic lesion to account for the pain, whereas a biochemical imbalance is responsible for primary dysmenorrhea. Primary dysmenorrhea affects 50 percent of post-pubescent women, and absenteeism among severe dysmenorrheics has been estimated to cost about several million lost working hours or billions of dollars annually.

The pain of dysmenorrhea originates in the uterus. Systemic administration of analgesic drugs generally by the oral route to the patient may not successfully relieve the condition in many women and the administration may be frequently limited by side effects. This failure is believed to be the result of a failure to deliver and achieve an effective dosage level of the analgesic to the muscle in the uterus.

There are various agents used to inhibit uterine contractions, such as, prostaglandin inhibitors, oxytocin antagonists, β-agonists, progestins (progesterone), nitric oxide substrates or donors. However, there exists a need for an effective, simple, and safe treatment of dysmenorrhea.

### SUMMARY OF THE INVENTION

Disclosed herein are methods, compositions, and devices for the treatment of uterine contractibility disorders, such as, dysmenorrhea.

In one aspect, there is provided a method for a prevention and/or treatment of dysmenorrhea or amelioration of a symptom thereof in a subject, comprising administering to said subject an effective amount of a compound of formula I:

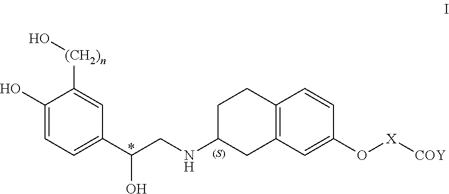

wherein
n is an integer selected from 1 or 2;
X is a C₁-C₆ alkylene group;
Y is —N(R)₂ wherein each R is independently selected from hydrogen or C₁-C₆ alkyl, or two R along with the nitrogen bound thereto join together to form a 3 to 7 membered heterocyclic ring optionally containing an oxygen atom; and
* represents a carbon atom in an R configuration, an S configuration, or a mixture thereof,
   a metabolite thereof, a prodrug thereof, or a pharmaceutically acceptable salt of any of the foregoing.

In one aspect, there is provided a method for a prevention and/or treatment of dysmenorrhea or amelioration of a symptom thereof in a subject, comprising administering to said subject an effective amount of a compound of formula V:

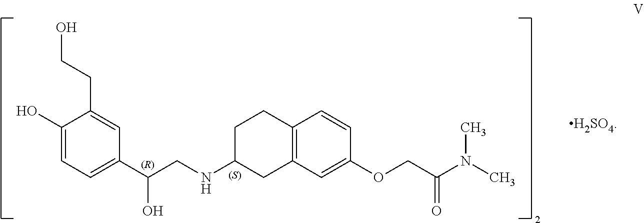

In another aspect, there is provided a method for a prevention and/or treatment of uterine contraction or cramping or amelioration of a symptom thereof in a subject, comprising administering to said subject an effective amount of a compound of formula I:

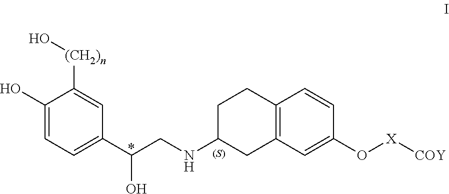

wherein
n is an integer selected from 1 or 2;
X is a C₁-C₆ alkylene group;
Y is —N(R)₂ wherein each R is independently selected from hydrogen or C₁-C₆ alkyl, or two R along with the nitrogen bound thereto join together to form a 3 to 7 membered heterocyclic ring optionally containing an oxygen atom; and
* represents a carbon atom in an R configuration, an S configuration, or a mixture thereof,
   a metabolite thereof, a prodrug thereof, or a pharmaceutically acceptable salt of any of the foregoing.

In another aspect, there is provided a method for uterine contraction or cramping or amelioration of a symptom thereof in a subject, comprising administering to said subject an effective amount of a compound of formula V:

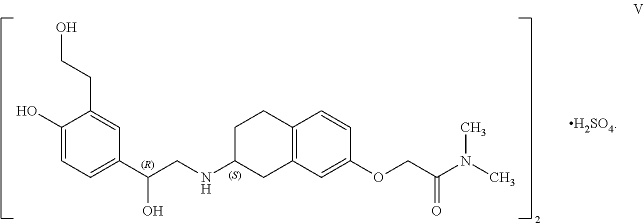

### DETAILED DESCRIPTION OF THE FIGURES

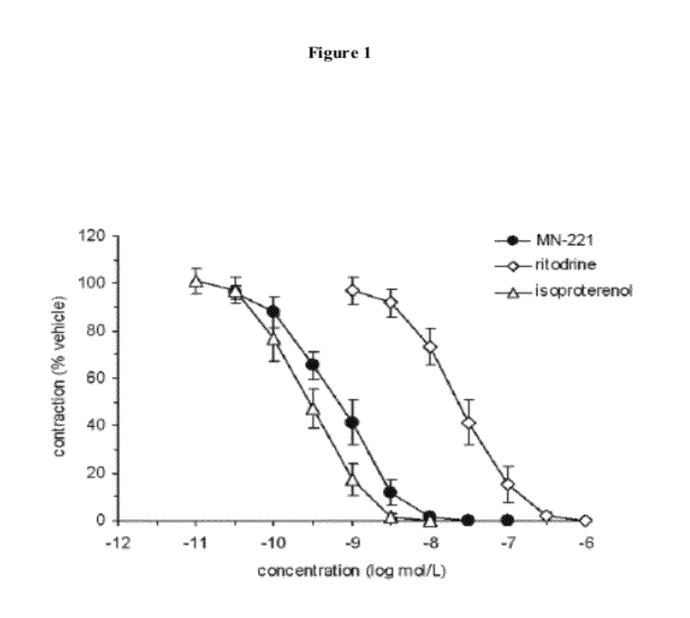

FIG. 1 illustrates the effects of MN-221, ritodrine hydrochloride, and isoproterenol bitartrate on spontaneous contractions of uterine muscle isolated from pregnant rats. The data represent the mean±standard error of 10 samples.

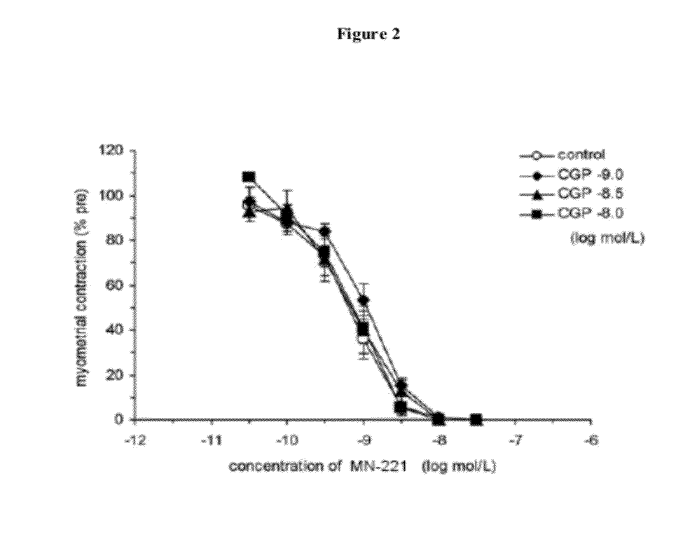

FIG. 2 illustrates the effect of CGP 20712A on inhibitory effect of MN-221 on uterine contraction. Each point represents the mean±standard error of 10 samples.

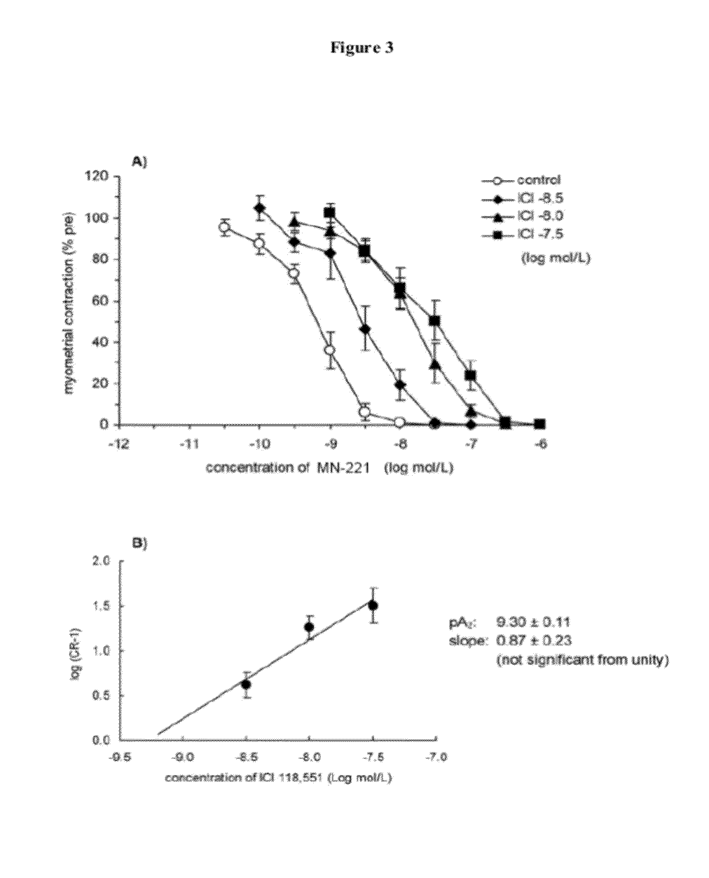

FIG. 3 illustrates the antagonistic effect of ICI 118,551 on inhibitory effect of MN-221 on uterine contraction. FIG. 3A) Concentration response curve: Each point represents the mean±standard error of 10 samples. FIG. 3B) Schild regression: Each point represents the results of 8 to 10 samples (a total of 28 samples).

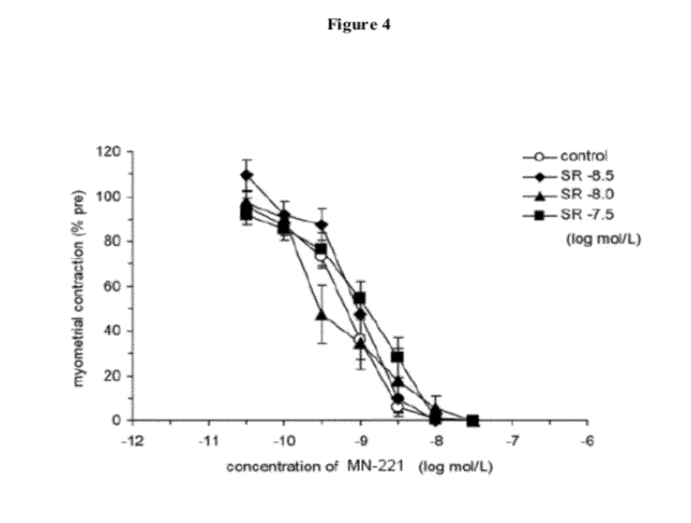

FIG. 4 illustrates the effect of SR 59230A on inhibitory effect of MN-221 on uterine contraction. Each point represents the mean±standard error of 10 to 12 samples.

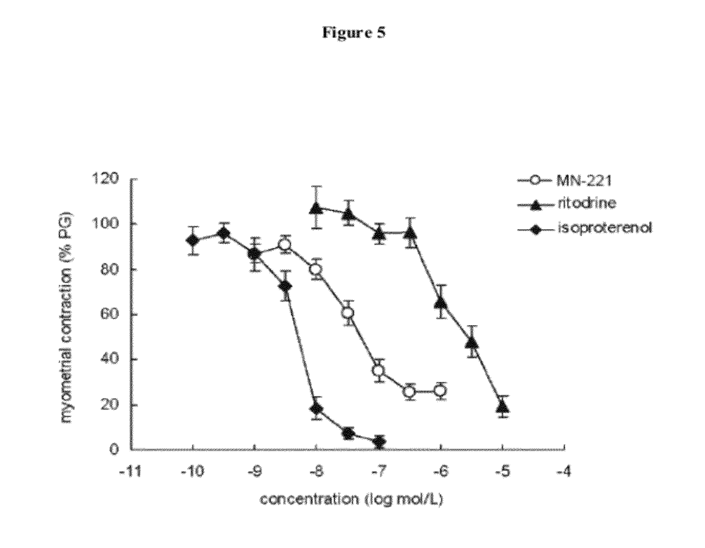

FIG. 5 illustrates the effect of MN-221, ritodrine hydrochloride, and isoproterenol bitartrate on PGF_{2α}-induced contraction of uterine muscle isolated from pregnant rats. Uterine contraction was induced with the addition of 5 μg/mL of PG F_{2α}. Each data point indicates a mean±standard error of 10 samples.

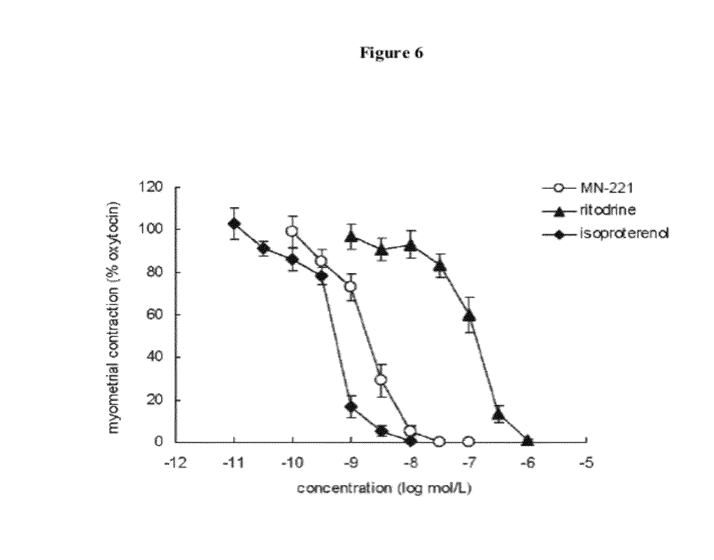

FIG. 6 illustrates the Effect of MN-221, ritodrine hydrochloride, and isoproterenol bitartrate on oxytocin-induced contraction of uterine muscle isolated from pregnant rats. Uterine contraction was induced with the addition of 1 mU/mL of oxytocin. Each data point indicates a mean±standard error of 10 samples.

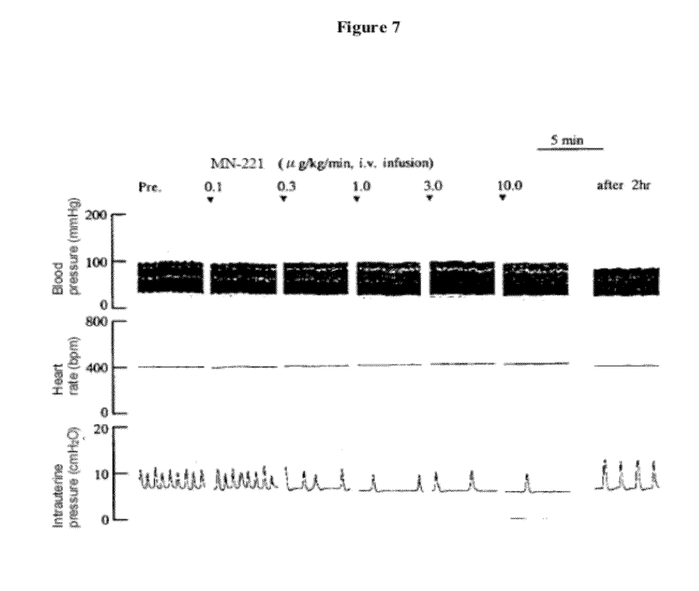

FIG. 7 illustrates the effects of MN-221 on uterine activity, heart rate, and blood pressure of anesthetized pregnant rats.

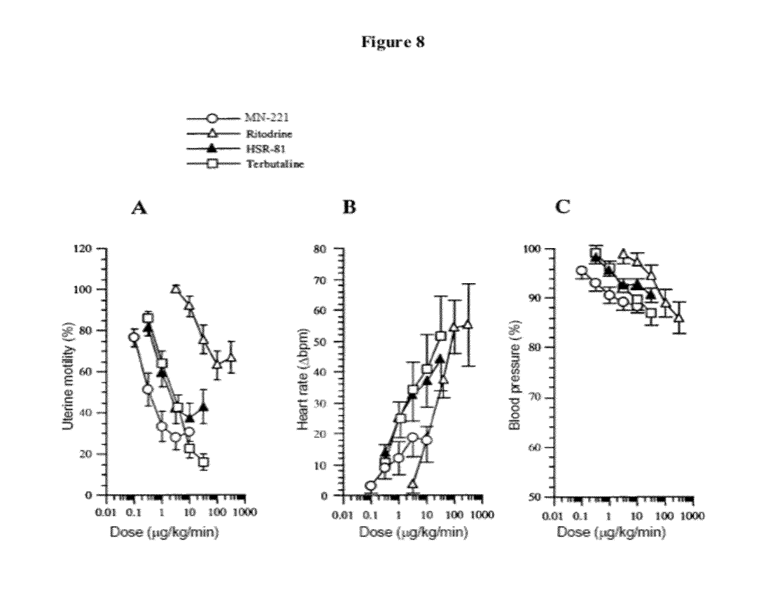

FIG. 8 illustrates the effects of MN-221 and other β2-adrenoceptor agonists on uterine activity of anesthetized pregnant rats (FIG. 8A), increases in heart rate of dam (FIG. 8B), and mean blood pressure of dam (FIG. 8C).

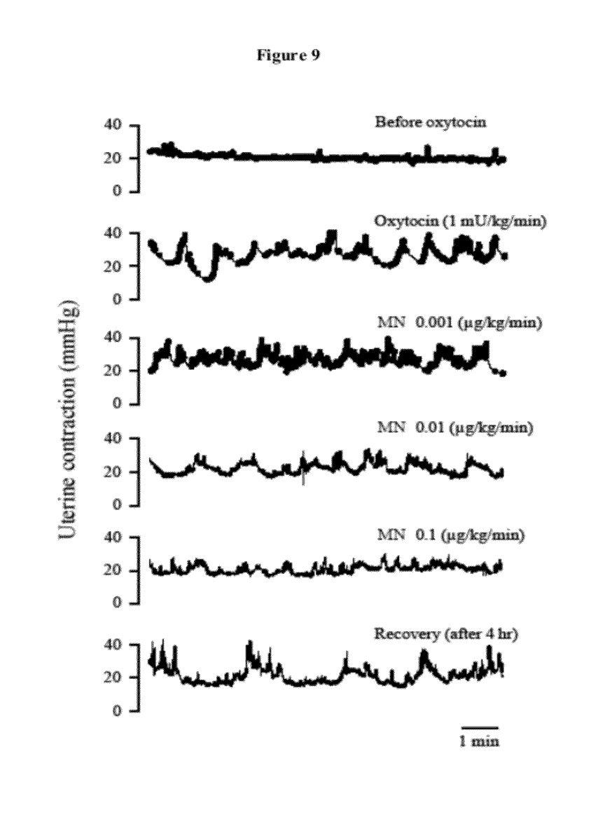

FIG. 9 illustrates representative recordings of the effect of MN-221 on the oxytocin-induced uterine contraction in the sheep.

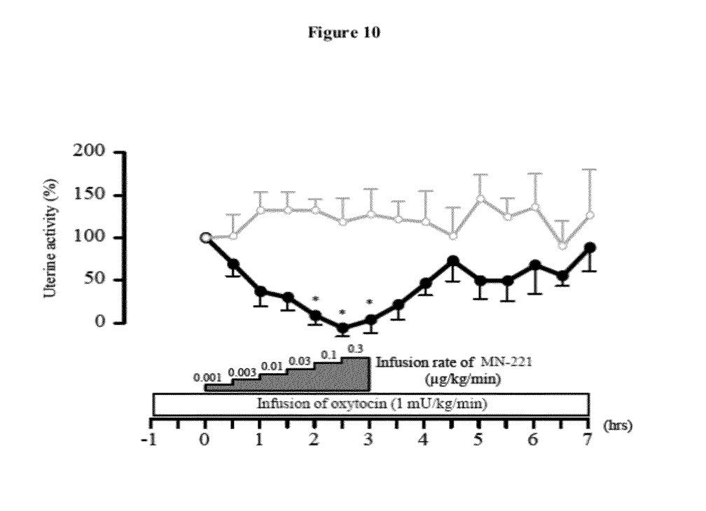

FIG. 10 illustrates comparison of changes over time in intrauterine pressure between the MN-221 group and the control group. MN-221 group is represented as closed circle; control group is represented as open circle; asterisk, P<0.05 compared with the pre-infusion value.

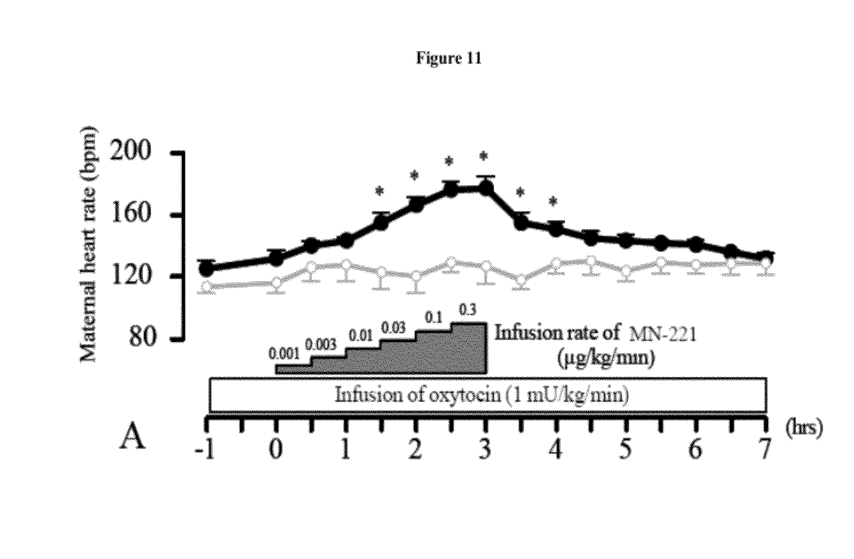

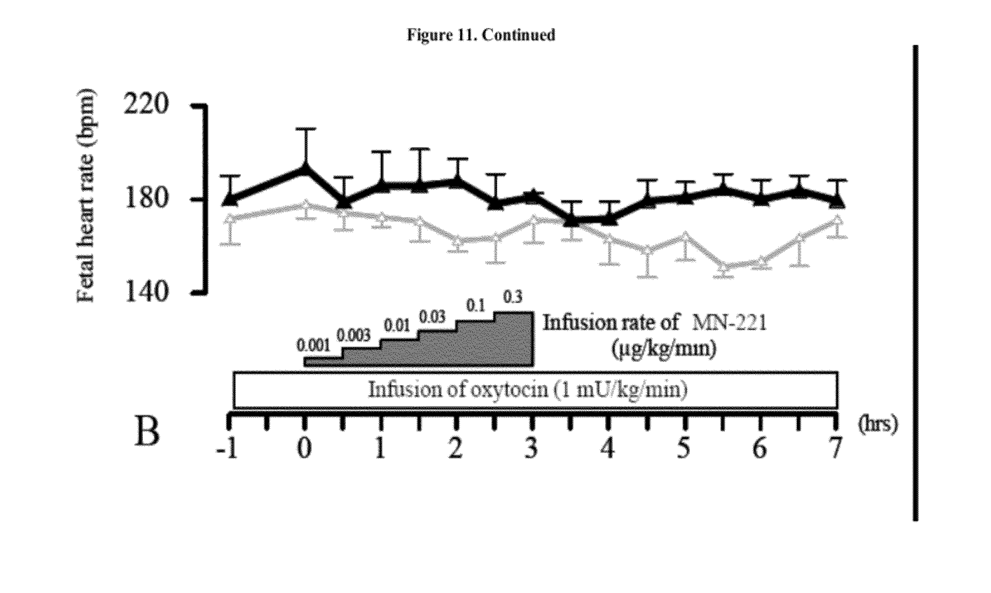

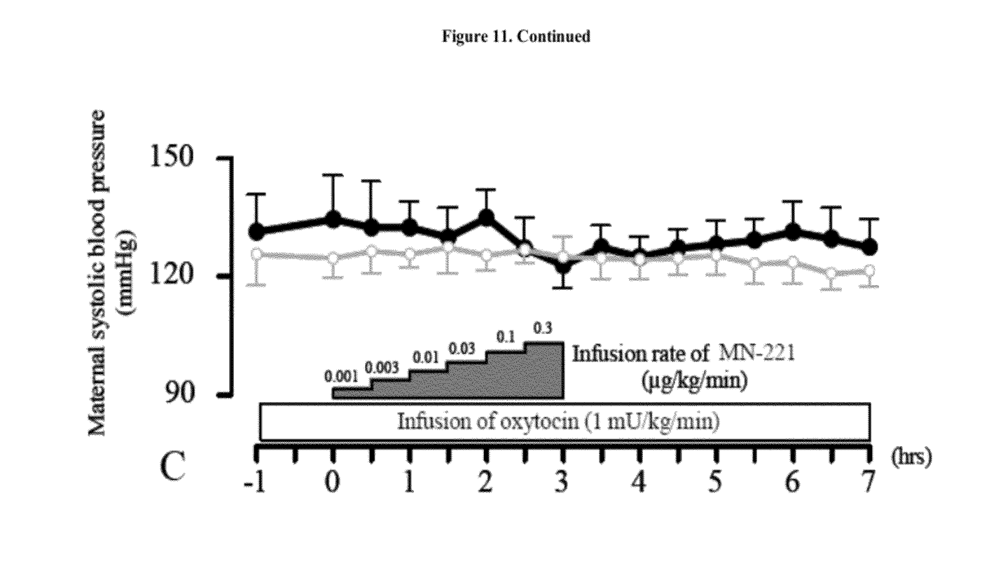

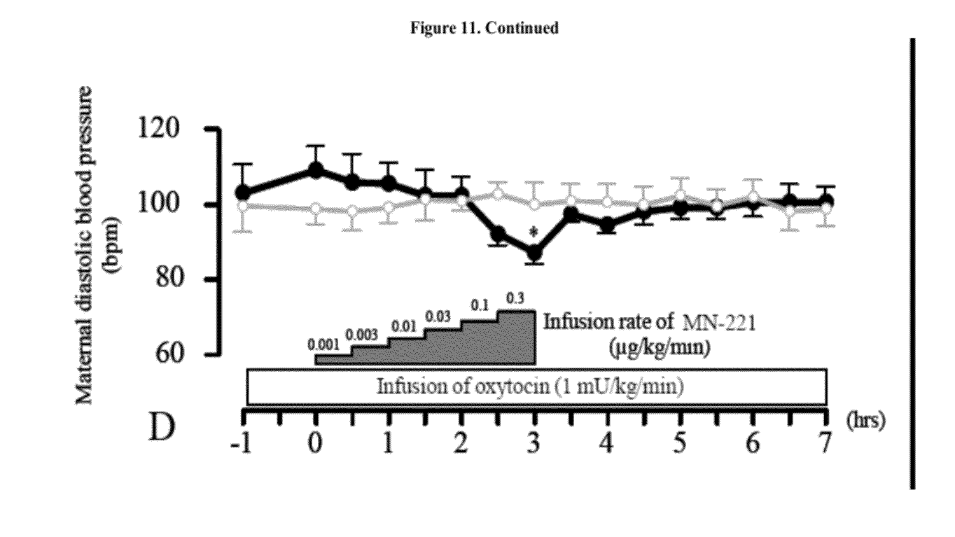

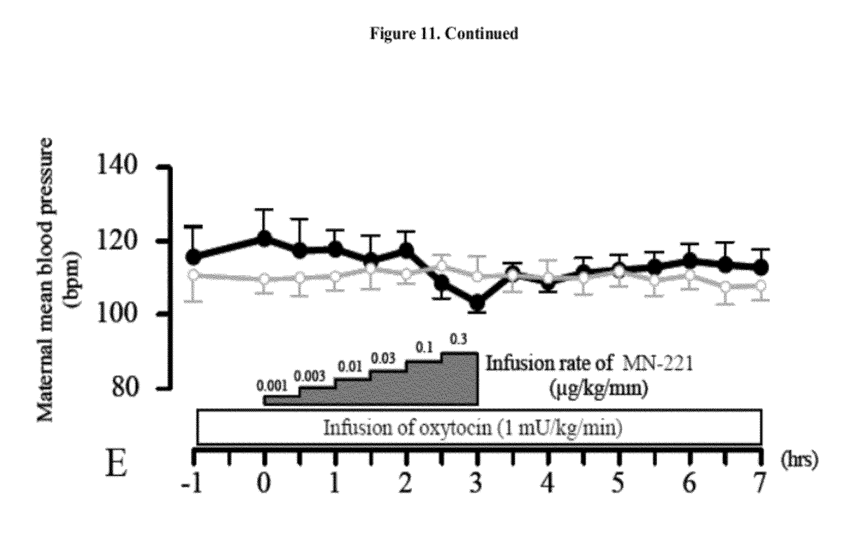

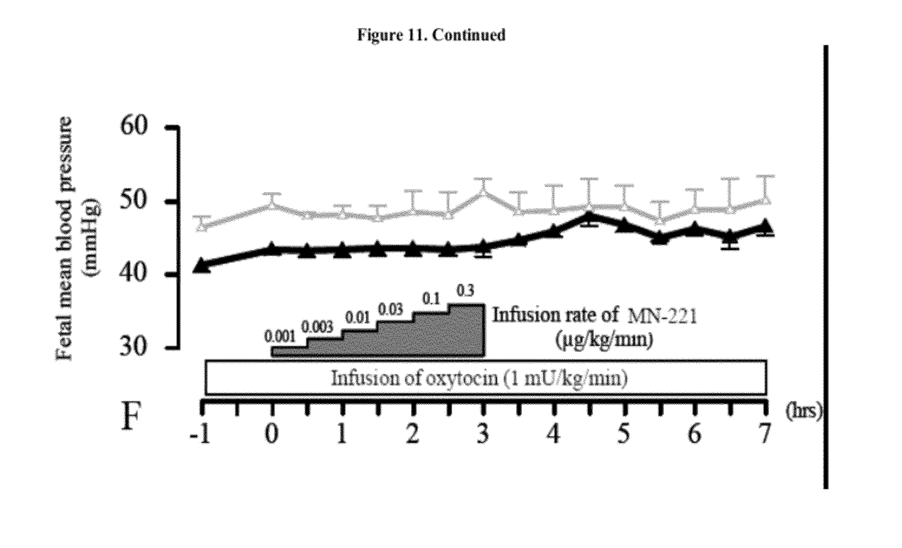

FIG. 11 illustrates comparison of changes over time in maternal and fetal heart rate and blood pressure between the MN-221 group and the control group during the experiment. FIG. 11A: maternal heart rate; FIG. 11B: fetal heart rate; FIG. 11C: maternal systolic blood pressure; FIG. 11D: maternal diastolic blood pressure; FIG. 11E: maternal mean blood pressure; and FIG. 11F: fetal mean blood pressure. MN-221 group is represented as closed circle & triangle; control group is represented as open circle & triangle; asterisk, P<0.05 compared with the pre-infusion value.

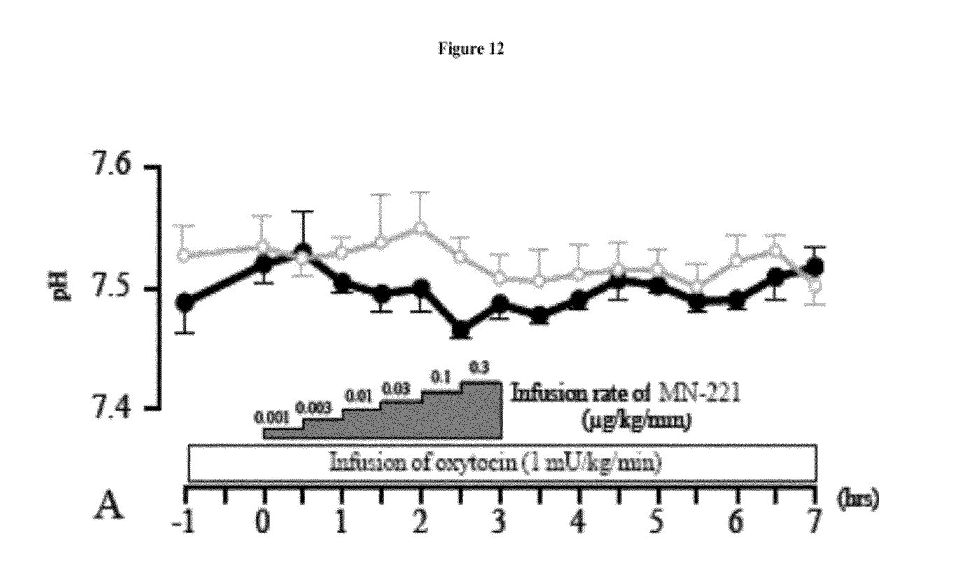

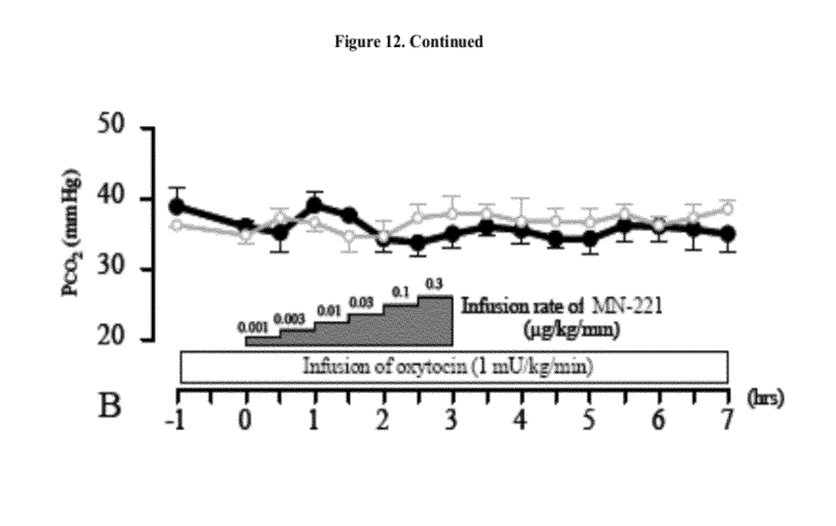

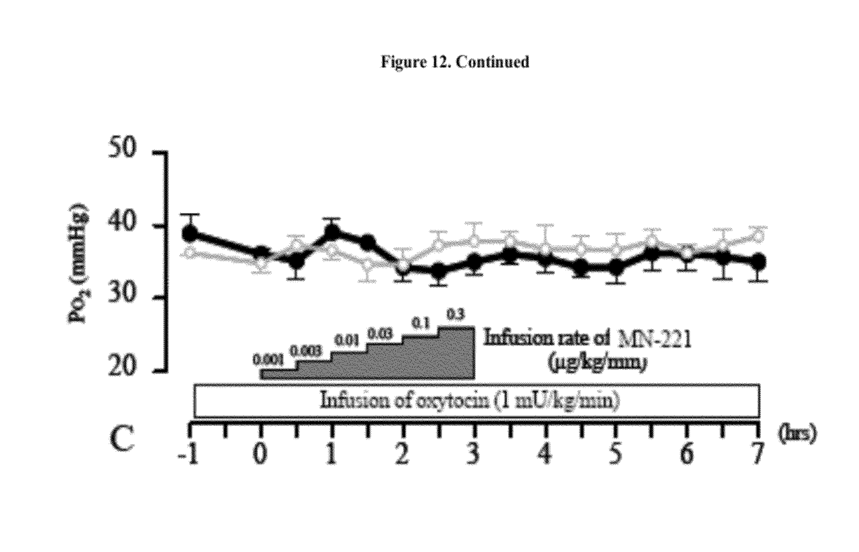

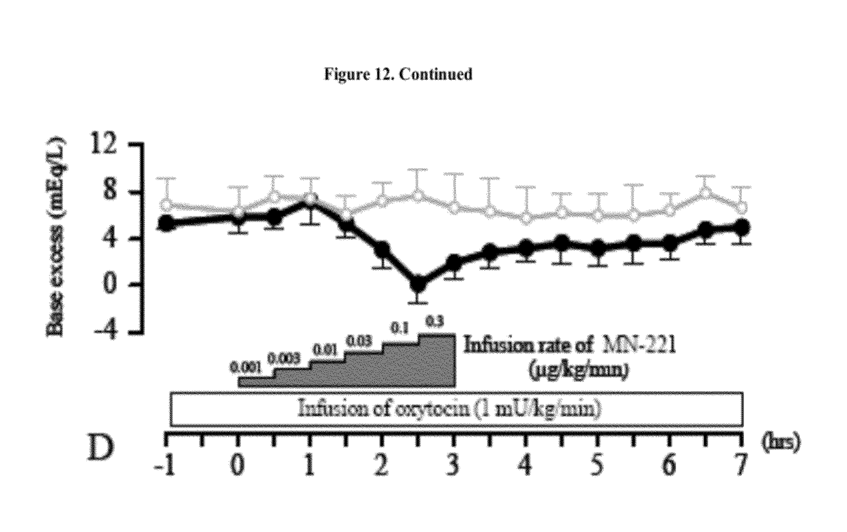

FIG. 12 illustrates comparison of changes over time in maternal respiratory parameters between the MN-221 group and the control group. FIG. 12A: pH; FIG. 12B: P_{CO2}; FIG. 12C: P_{O2}; and FIG. 12D: base excess. MN-221 group is represented as closed circle, and control group is represented as open circle.

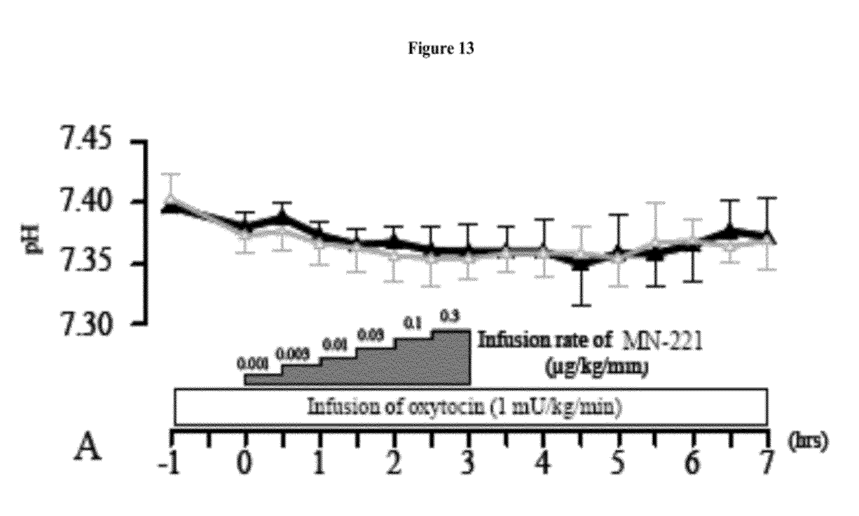

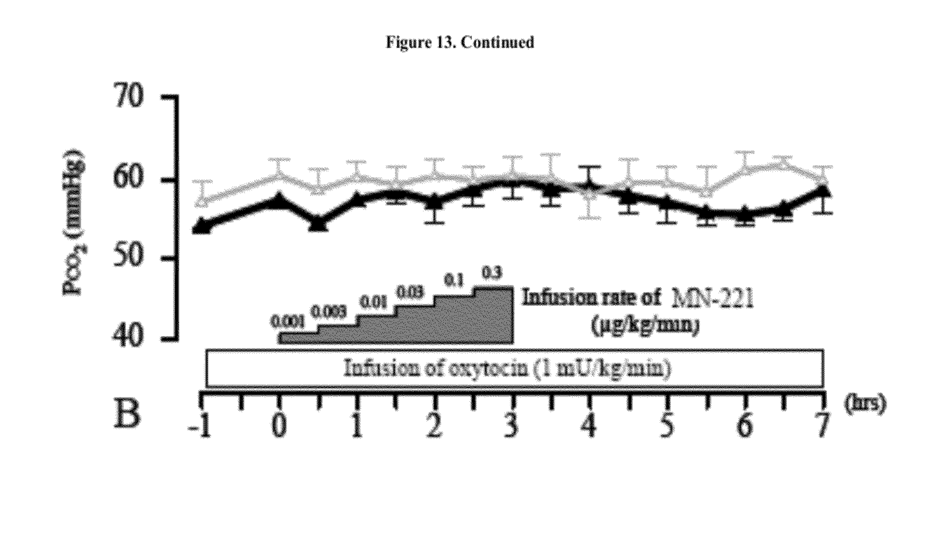

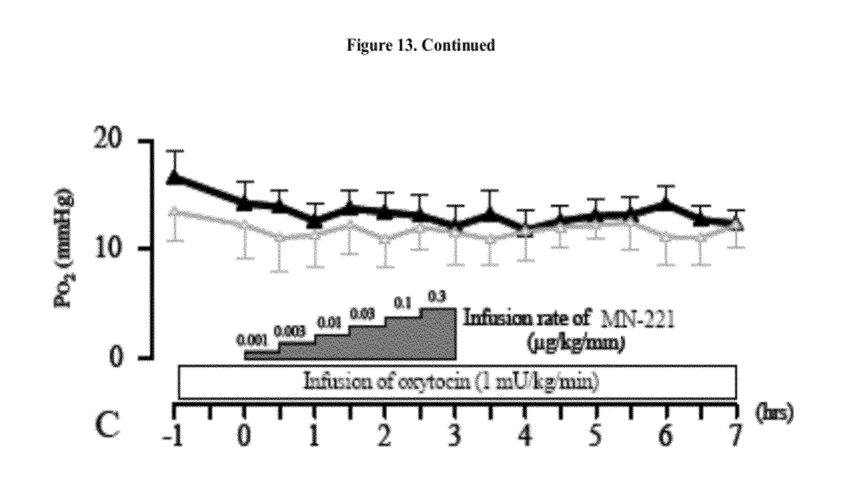

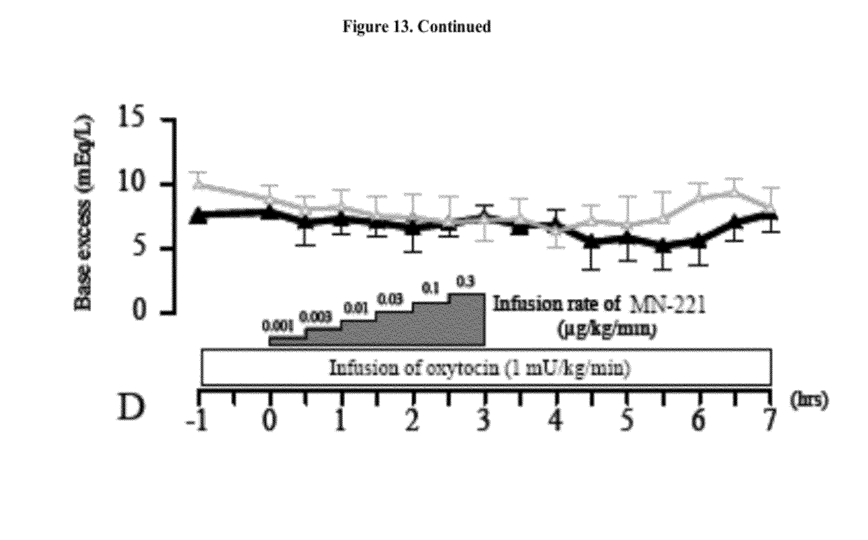

FIG. 13 illustrates comparison of changes over time in fetal respiratory parameters between the MN-221 group and the control group. FIG. 13A: pH, FIG. 13B: P_{C02}, FIG. 13C: P_{O2}, and FIG. 13D: base excess. MN-221 group is represented as closed triangle and control group is represented as open triangle.

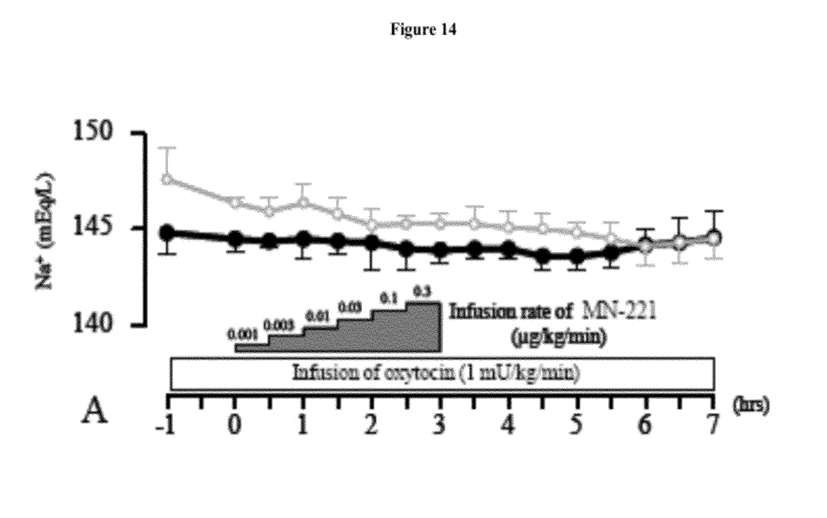

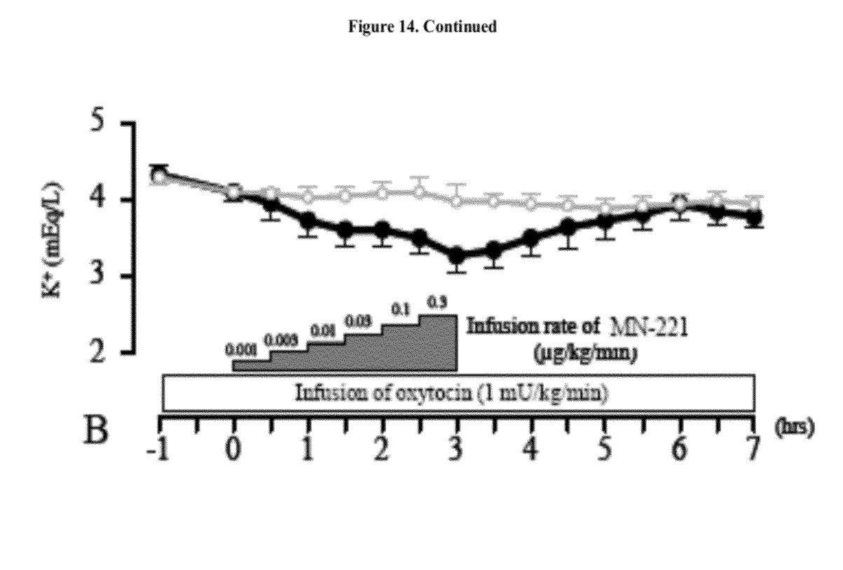

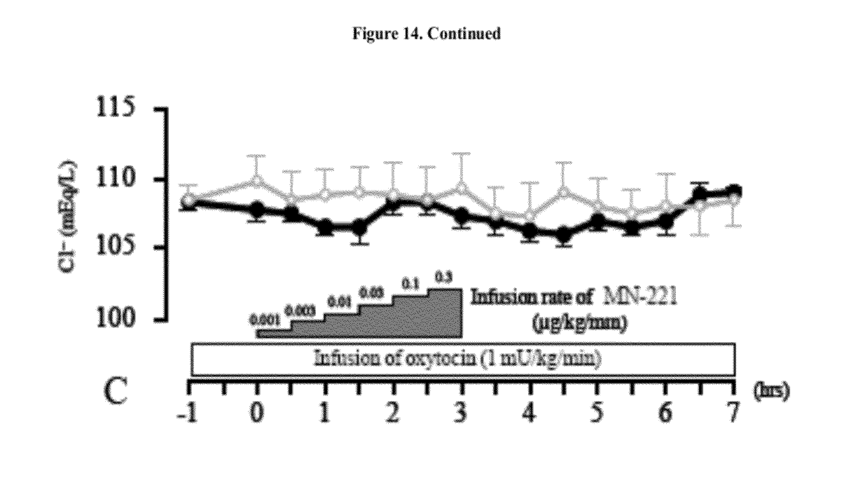

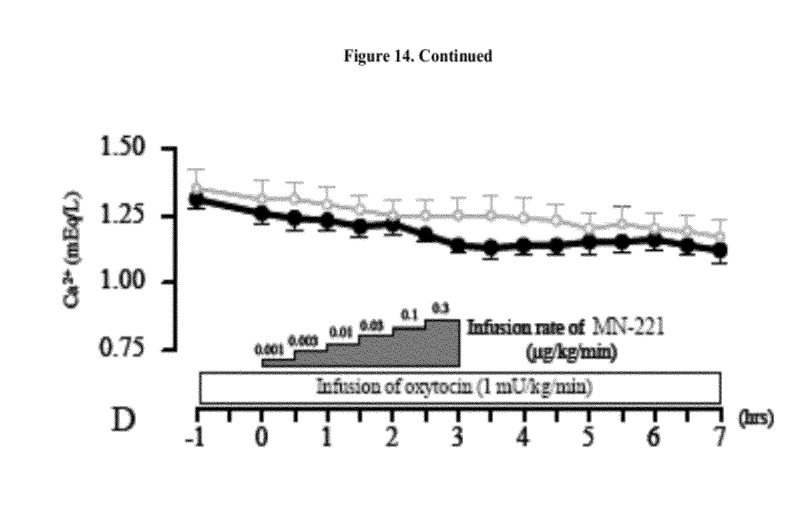

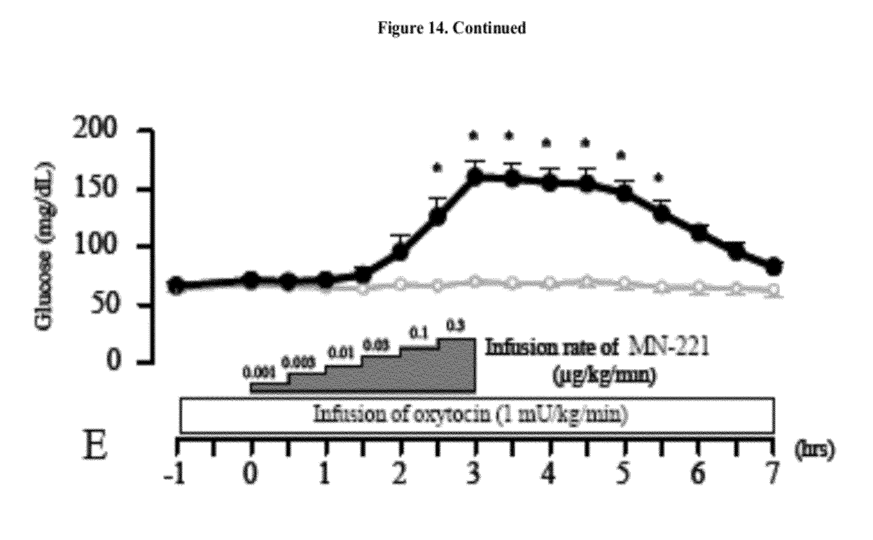

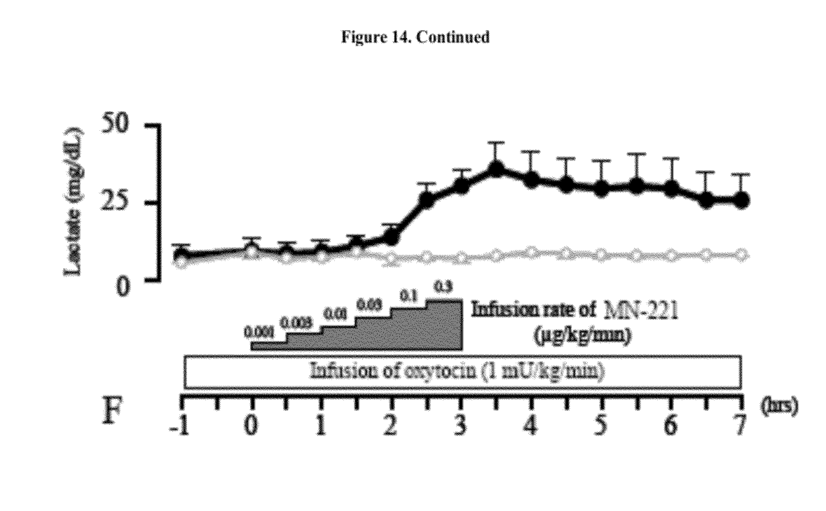

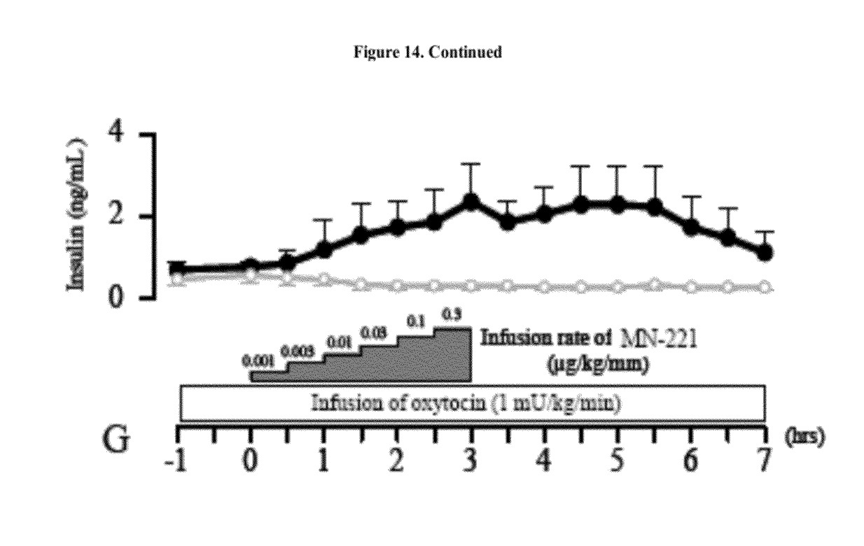

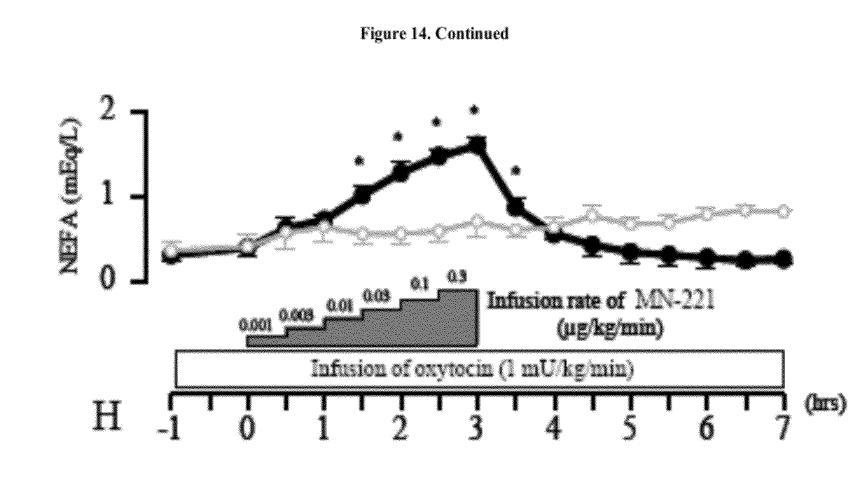

FIG. 14 illustrates comparison of changes over time in maternal metabolic parameters between the MN-221 group and the control group. FIG. 14A: blood Na⁺; FIG. 14B: blood K⁺; FIG. 14C: blood Cl⁻; FIG. 14D: blood Ca²⁺; FIG. 14E: plasma glucose; FIG. 14F: blood lactate; FIG. 14G: plasma insulin; and FIG. 14H: plasma NEFA levels. MN-221 group is represented as closed circle and the control group is represented as open circle. Asterisk, P<0.05 compared with the pre-infusion value.

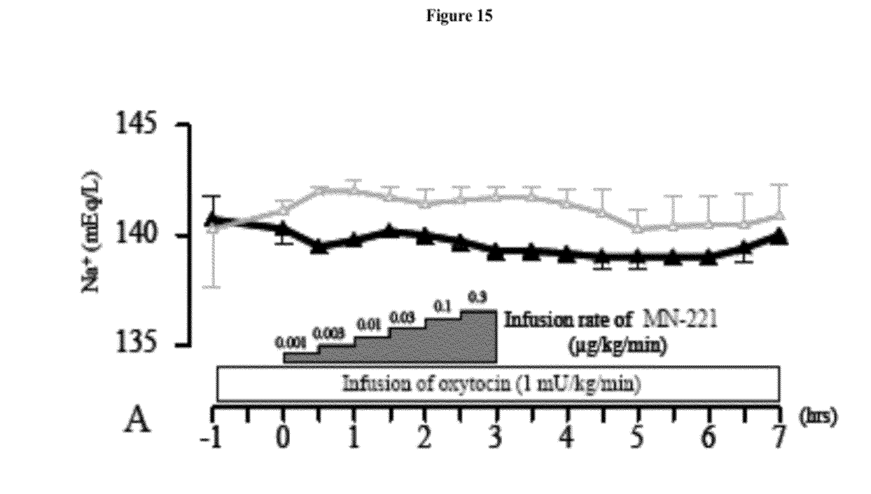

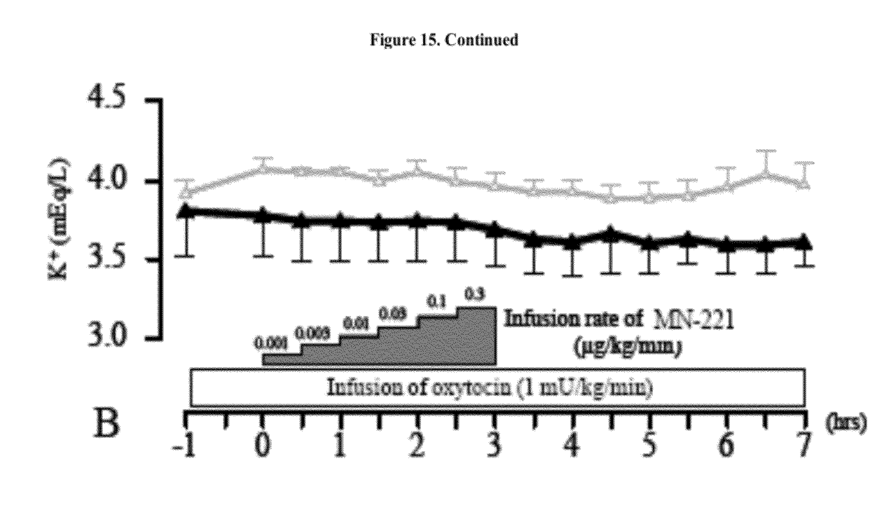

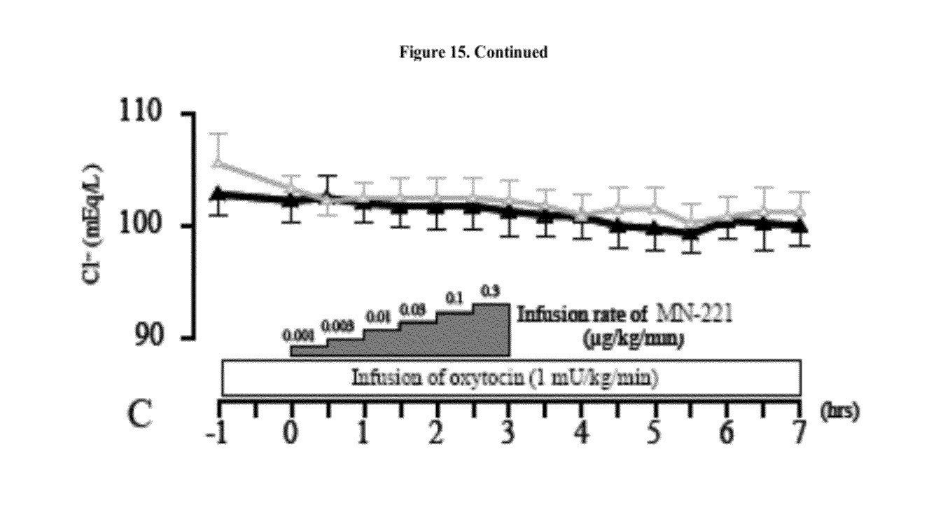

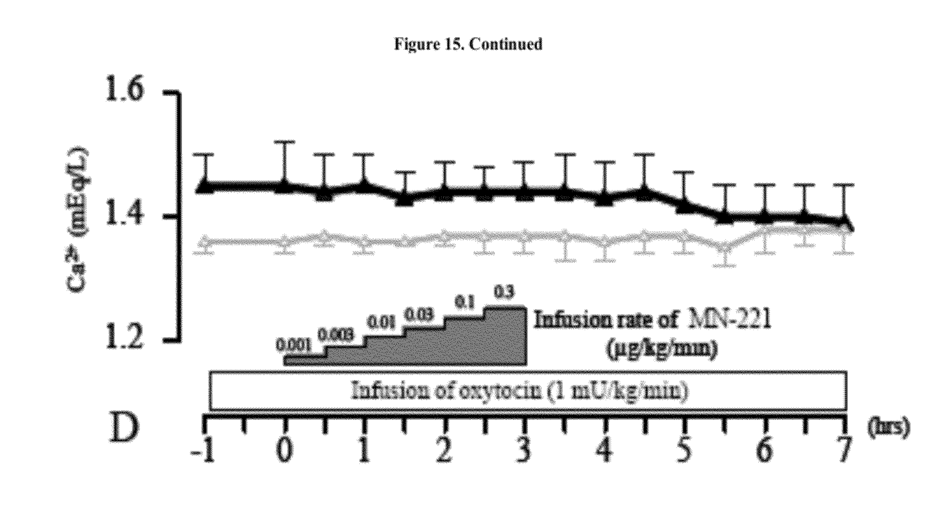

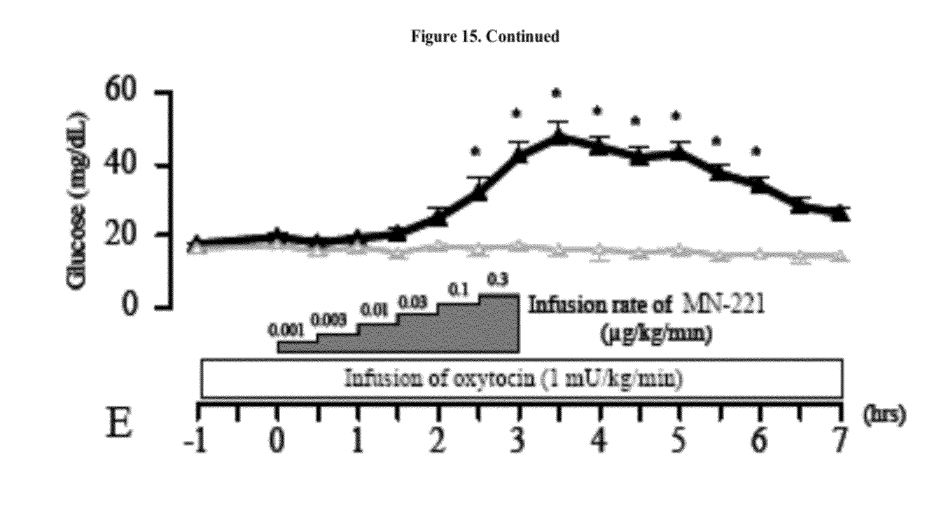

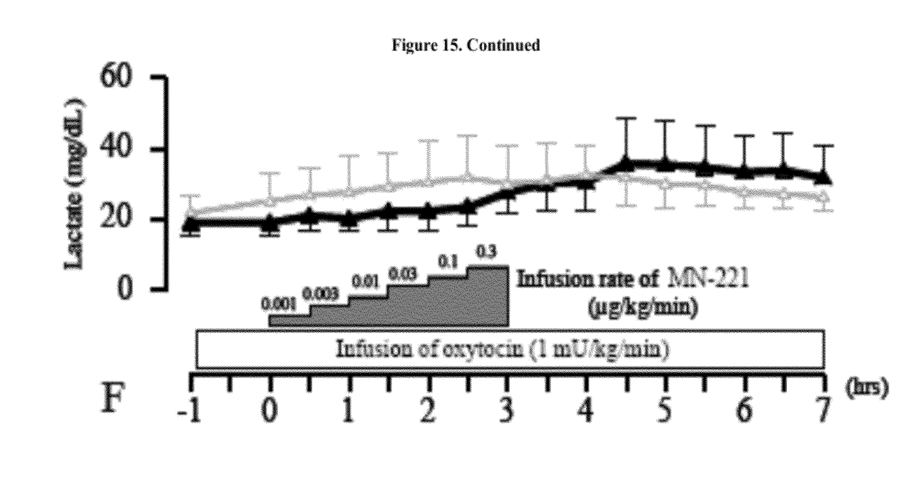

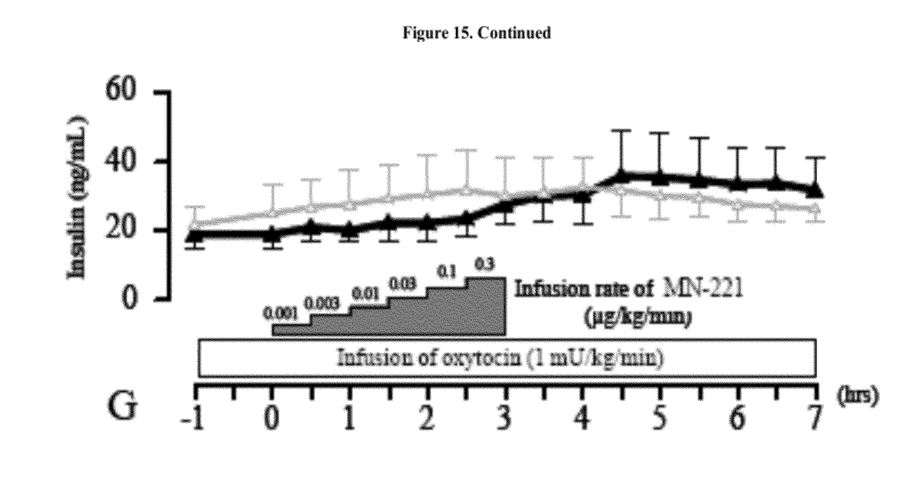

FIG. 15 illustrates comparison of changes over time in fetal metabolic parameters between the MN-221 group and the control group. FIG. 15A: blood Na⁺; FIG. 15B: blood K⁺; FIG. 15C: blood Cl⁻; FIG. 15D: blood Ca²⁺; FIG. 15E: plasma glucose; FIG. 15F: blood lactate; and FIG. 15G: plasma insulin. MN-221 group is represented as closed triangle and the control group is represented as open triangle. Asterisk, P<0.05 compared with the pre-infusion value.

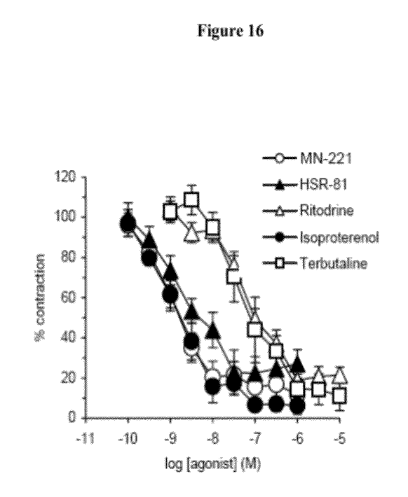

FIG. 16 illustrates the effects of MN-221 and other β2-adrenoceptor agonists on spontaneous contractions of uterine muscles isolated from pregnant rabbits.

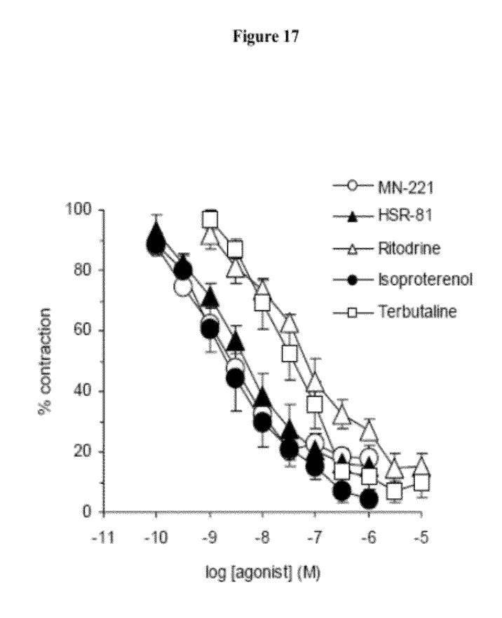

FIG. 17 illustrates the effects of MN-221 and other β2-adrenoceptor agonists on oxytocin-induced contractions of uterine muscles isolated from pregnant rabbits.

### DETAILED DESCRIPTION OF THE INVENTION

### 1. Definitions

It must be noted that as used herein, and in the appended claims, the singular forms “a,” “an,”, and “the” include plural references unless the context clearly dictates otherwise.

Unless defined otherwise, all technical, and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology, and pharmacology, within the skill of the art. Such techniques are explained fully in the literature. (See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; D. M. Weir, and C. C. Blackwell, eds. (1986) Handbook of Experimental Immunology, Vols. I-IV, Blackwell Scientific Publications; Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press; Newton & Graham eds. (1997) PCR (Introduction to Biotechniques Series), 2nd ed., Springer Verlag).

An “administration” or “administering,” refers to the delivery of a medication, such as the composition used according to the invention to an appropriate location of the subject or in vitro, where a desired effect is achieved. Non-limiting examples include topical, oral, parenteral, direct application to target area or proximal areas on the skin, or applied transdermally such as a patch. Various physical and/or mechanical technologies are available to permit the sustained or immediate release of the composition after administration.

A “C₁-C₆ alkyl” refers to saturated monovalent hydrocarbyl groups having from 1 to 6 carbon atoms, more particularly from 1 to 5 carbon atoms, and even more particularly 1 to 3 carbon atoms. This term is exemplified by groups such as methyl, ethyl, n-propyl, iso-propyl, n-butyl, t-butyl, n-pentyl, and the like.

A “C₁-C₆ alkylene” refers to divalent saturated aliphatic hydrocarbyl groups having from 1 to 6 carbon atoms and, in some embodiments, from 1 to 3 carbon atoms. The alkylene groups include branched and straight chain hydrocarbyl groups. Examples include methylene (—CH₂—), ethylene, propylene, 2-methypropylene, pentylene, and the like.

A “compound” herein refers to a compound used according to the invention, a pharmaceutically acceptable salt thereof, a metabolite thereof, a prodrug thereof, a pharmaceutically acceptable salt of the metabolite thereof, or a pharmaceutically acceptable salt of the prodrug thereof. The compounds include stereoisomeric forms and the tautomeric forms of the compounds.

“Comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

An “effective amount” or a “therapeutically effective amount” is an amount sufficient to effect beneficial or desired results, e.g., alleviation, amelioration, palliation or elimination of one or more manifestations of dysmenorrhea in the subject. The full therapeutic effect may occur in one dose; may not necessarily occur by administration of one dose (or dosage); and may occur only after administration of a series of doses. Thus, a therapeutically effective amount may be administered in one or more administrations, applications or dosages.

A “heterocycle” or “heterocyclic” refers to a saturated or unsaturated (but not aromatic) group having a single ring or multiple condensed rings, from 3 to 6 carbon atoms, and from 1 to 4 hetero atoms selected from the group consisting of nitrogen or oxygen within the ring wherein, in fused ring systems, one or more of the rings can be aryl or heteroaryl provided that the point of attachment is at the heterocycle. The nitrogen ring atoms can optionally be oxidized to provide for the N-oxide derivatives. Examples of heterocycles include, but are not limited to, azetidine, pyrrole, imidazole, pyrazole, pyridine, pyrazine, pyrimidine, pyridazine, etc.

A “metabolite” refers to any substance that is produced as an intermediate or a product after the metabolism of the compound used according to the invention. Examples of metabolites include, but are not limited to, acid metabolized from the amide moiety, amine metabolized from the substituted amide moiety, alcohol metabolized from alkoxy moiety, and the like. A carboxylic acid, representative of a metabolite, is described in U.S. Pat. No. 6,136,852, the disclosure of which is incorporated herein by reference in its entirety.

A “subject” or “patient” is a female mammal, including a human. Non-human animals subject to diagnosis or treatment include, for example, murine, such as rats, mice, canine, such as dogs, leporids, such as rabbits, livestock, sport animals, and pets.

A “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

A “pharmaceutically acceptable salt” refers to pharmaceutically acceptable salts of a compound, which salts are derived from a variety of organic, and inorganic counter ions well known in the art. When the molecule contains a basic functionality, salts include, by way of example only, sodium, potassium, calcium, magnesium, ammonium, tetraalkylammonium, and the like. When the molecule contains a basic functionality, salts of organic or inorganic acids include, such as hydrochloric, sulfuric, phosphoric, acetic, citric, oxalic, malonic, salicyclic, malic, gluconic, fumaric, succinic, ascorbic, maleic, methanesulfonic acid, etc.

A “prodrug”, as used herein, refers to any covalently bonded carrier which releases the active parent drug in vivo when such prodrug is administered to a subject. Prodrugs of a compound are prepared by modifying functional groups present in the compounds in such a way that the bonds are cleaved, either in routine manipulation or in vivo, to the parent compounds. Prodrugs include, but are not limited to, compounds wherein hydroxyl or amine groups are bonded to any group that, when administered to a subject, cleave to form a free hydroxyl or amino, group, respectively. Examples of prodrugs include, but are not limited to, acetate, formate, benzoate and phosphate ester derivatives of hydroxyl functional groups, especially the hydroxyl group on the phenyl ring of formula I, and acetyl and benzoyl derivatives of amine functional groups in the compounds of the invention and the like.

A “treating,” “treatment” and the like refer to obtaining a desired pharmacologic and/or physiologic effect. The effect can be prophylactic in terms of completely or partially preventing a disease or disorder or sign or symptom thereof, and/or can be therapeutic in terms of a partial or complete cure for a disorder and/or adverse effect attributable to the disorder. Examples of “treatment” include but are not limited to: preventing a disease from occurring in a subject that may be predisposed or at risk of a disease, but has not yet been diagnosed as having it; inhibiting a disease, i.e., arresting its development; and/or relieving or ameliorating the symptoms of disease or reducing the likelihood of recurrence of the disease, such as dysmenorrhea. As is understood by those skilled in the art, “treatment” can include systemic amelioration of the symptoms associated with the pathology and/or a delay in onset of symptoms.

### 2. Methods Of The Invention

In one aspect, there is provided a method for a prevention and/or treatment of dysmenorrhea or amelioration of a symptom thereof in a subject, comprising administering to the subject an effective amount of a compound, as provided herein.

Dysmenorrhea is a condition that refers to the pain or discomfort associated with menstruation in female subjects. Dysmenorrhea may be classified as primary or secondary. Primary dysmenorrhea is a severe and frequent menstrual cramping caused by severe and abnormal uterine contractions. Secondary dysmenorrhea is a painful menstrual period caused by another medical condition present in the body (e.g., pelvic inflammatory disease, endometriosis). Endometriosis is a condition in which tissue that appears and acts like endometrial tissue becomes implanted outside the uterus, typically on other reproductive organs inside the pelvis or in the abdominal cavity, resulting in internal bleeding, infection, and pelvic pain. Other possible causes of secondary dysmenorrhea include, but are not limited to, pelvic inflammatory disease (PID), pelvic congestion syndrome, pelvic infection, cervical stenosis, uterine fibroids, adenomyosis, abnormal pregnancy (i.e., miscarriage, ectopic), and infection, tumors, or polyps in the pelvic cavity.

The common symptoms of dysmenorrhea resemble symptoms of other conditions or medical problems, such as, but are not limited to, cramping in the lower abdomen; pain in the lower abdomen; low back pain; pain radiating down the legs; nausea; vomiting; diarrhea; fatigue; weakness; fainting; and headaches.

The dosage and the regimen for the treatment for dysmenorrhea using the compositions and methods of the invention can depend on age, overall health, and medical history; extent of the condition; cause of the condition (primary or secondary); and tolerance for specific medications, procedures, or therapies.

Accordingly, in some embodiments, there is provided a method for a prevention and/or treatment of dysmenorrhea or amelioration of a symptom thereof in a subject, comprising administering to said subject an effective amount of a compound of formula I:

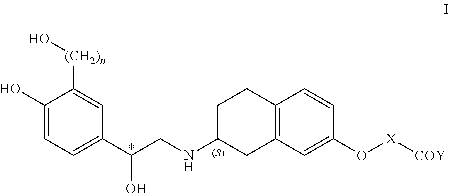

wherein
n is an integer selected from 1 or 2;
X is a C₁-C₆ alkylene group;
Y is —N(R)₂ wherein each R is independently selected from hydrogen or C₁-C₆ alkyl, or two R along with the nitrogen bound thereto join together to form a 3 to 7 membered heterocyclic ring optionally containing an oxygen atom; and
* represents a carbon atom in an R configuration, an S configuration, or a mixture thereof,
   a metabolite thereof, a prodrug thereof, or a pharmaceutically acceptable salt of any of the foregoing.

In some embodiments, there is provided a method for a prevention and/or treatment of dysmenorrhea or amelioration of a symptom thereof in a subject, comprising administering to said subject an effective amount of a compound of formula II:

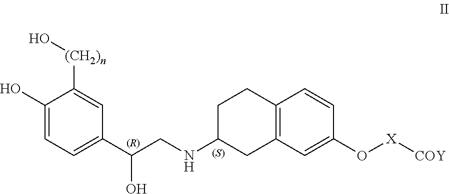

wherein
n is an integer selected from 1 or 2;
X is a C₁-C₆ alkylene group; and
Y is —N(R)₂ wherein each R is independently selected from hydrogen or C₁-C₆ alkyl, or two R along with the nitrogen bound thereto join together to form a 3 to 7 membered heterocyclic ring optionally containing an oxygen atom;
a metabolite thereof, a prodrug thereof, or a pharmaceutically acceptable salt of any of the foregoing.

In some embodiments, there is provided a method for a prevention and/or treatment of dysmenorrhea or amelioration of a symptom thereof in a subject, comprising administering to said subject an effective amount of a compound of formula III:

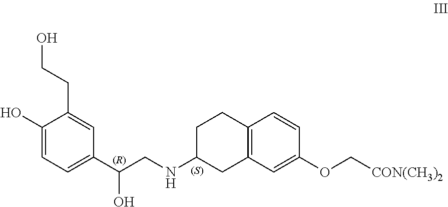

a metabolite thereof, a prodrug thereof, or a pharmaceutically acceptable salt of any of the foregoing.

In some embodiments, there is provided a method for a prevention and/or treatment of dysmenorrhea or amelioration of a symptom thereof in a subject, comprising administering to said subject an effective amount of a metabolite of formula IV:

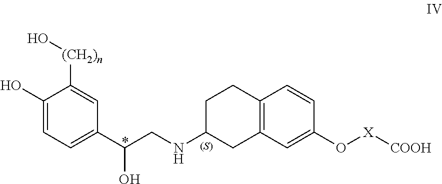

wherein
n is an integer selected from 1 or 2;
X is a C₁-C₆ alkylene group; and
* represents a carbon atom in an R configuration, an S configuration, or a mixture thereof,
or a pharmaceutically acceptable salt thereof.

In some embodiments, there is provided a method for a prevention and/or treatment of dysmenorrhea or amelioration of a symptom thereof in a subject, comprising administering to said subject an effective amount of a compound of formula V:

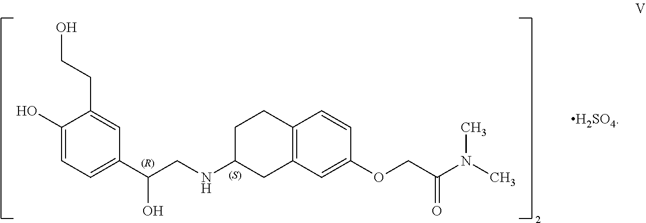

In one aspect, the present invention provides a method for treating a human female suffering from dysmenorrhea.

In some embodiments, the methods provided herein further comprise administering to the subject a drug selected from the group consisting of non-steroidal anti-inflammatory drugs, anti-prostaglandins, COX-2 inhibitors, local anesthetics, calcium channel blockers, potassium channel blockers, leukotriene blocking agents, smooth muscle inhibitors, vasodilators, and drugs capable of inhibiting dyskinetic muscle contraction.

Non-limiting examples of non-steroidal anti-inflammatory drugs suitable for use in the method of the invention include, but are not limited to, aspirin, ibuprofen, indomethacin, phenylbutazone, bromfenac, fenamate, sulindac, nabumetone, ketorolac, and naproxen. Examples of local anesthetics include, but are not limited to, lidocaine, mepivacaine, etidocaine, bupivacaine, 2-chloroprocaine hydrochloride, procaine, and tetracaine hydrochloride. Examples of calcium channel blockers include, but are not limited to, diltiazem, israpidine, nimodipine, felodipine, verapamil, nifedipine, nicardipine, and bepridil. Examples of potassium channel blockers include, but are not limited to, dofetilide, E-4031, almokalant, sematilide, ambasilide, azimilide, tedisamil, RP58866, sotalol, piroxicam, and ibutilide. Vasodilators, which are believed to relieve muscle spasm in the uterine muscle, include, but are not limited to, nitroglycerin, isosorbide dinitrate and isosorbide mononitrate. Examples of COX-2 inhibitors include, but are not limited to, celecoxib, meloxicam and flosulide. A synergistic effect may be achieved by using a combination of the compound used according to the invention (e.g., those encompassed by formulas I, II, III, IV, V, and metabolites, isomers, and prodrugs of each thereof) with the drugs recited above.

In some embodiments, the compound used according to the invention and optionally the above recited drug is in combination with a biocompatible excipient provided herein. In some embodiments, the compound used according to the invention is present in an amount sufficient to attain a therapeutically effective amount of the compound in the uterine muscle of the subject upon administration. In some embodiments, the drug is absorbable through the vaginal mucosa and thereby transmitted via venous and lymphatic channels to the uterus.

In practicing the invention, a subject need not wait until the onset of menses and the occurrence of pain to begin treatment. The present invention comprises administration of the compound or the composition as soon as the subject realizes that she is nearing the onset of menses, for example within a day or two. The method disclosed herein prevent the process of dyskinetic contractions from occurring, including treating them once the contractions have already begun.

In some embodiments, the compositions provided herein can treat dysmenorrhea and its dyskinetic contractions, without interfering with the normal contractions and bleeding during menstruation. Dysmenorrhea involves dyskinetic contractions, which are erratic and abnormal with an increase in the amplitude and frequency of contraction. Dysmenorrhea includes, without limitation, antegrade contractions (fundus to cervix), retrograde contractions (cervix to fundus), and non-functional fibrillations. In some embodiments, the composition of the present invention treats dysmenorrhea by selective action on the dyskinetic contractions without preventing the normal, regularized contractions necessary for menstruation. As menstrual blood does not clot, normal, regularized contractions are helpful to stop the bleeding. If there are no contractions, then the patient may not stop bleeding and may hemorrhage. In some embodiments, the compound of the present invention interferes with the dyskinetic contractions causing dysmenorrhea, without stopping contractions entirely.

In some embodiments, the compositions and/or devices of the invention or the compositions and/or devices used according to the invention are applied several hours before or just after onset of menstruation in order to treat or prevent dysmenorrhea. The treatment would continue for a few hours up to 6 days, as needed, to alleviate and prevent painful menstruation and symptoms such as nausea, fatigue, diarrhea, lower backache, and headache.

In some embodiments, the administration of the compound according to the invention to the subject results in reduced, negligible, or no adverse side effects. Typically, the side effects of common β-adrenergic agonists include, but are not limited to, cardiovascular such as palpitations, peripheral tremors, high heart rate, and low blood pressure; pulmonary edema and hypoglycemia; aggravation of preexisting diabetes and keto acidosis; tremors; nervousness; increased heart rate; palpitations; dizziness; headaches; drowsiness; vomiting; nausea; sweating; muscle cramps; and ECG changes. In some embodiments, the use of the compounds according to the invention reduces or eliminates one or more of the above-noted side effects. It is important to note that such reduced, negligible, or lack of adverse side effects may be especially manifest when comparing the outcomes using the compounds according to the invention with outcomes using other β-adrenergic agonists, including but not limited to one or more of HSR-81, terbutaline, ritodrine, isoproterenol, or pharmaceutically acceptable salts thereof.

Accordingly, in the methods provided herein, the administration of the compounds reduce the incidence of one or more adverse side effects in the subject. In some embodiments, the number of incidences of the one or more of adverse side effects in the subject is reduced with the administration of the compound according to the invention as compared to the number of such incidences, which would have been observed in the subject with the administration of terbutaline, ritodrine, or meluadrine. In some embodiments, the β-adrenergic agonist is terbutaline, ritodrine hydrochloride, or HSR-81. In some embodiments, the administration of a compound according to the invention reduces the incidence of one or more adverse side effects in the subject as compared to terbutaline. In some embodiments, the number of incidences of increased heart rate, decrease in mean blood pressure, or both in the subject after the administration of the compound according to the invention is reduced compared to the number of such incidences, which would have been observed in the subject with the administration of terbutaline.

The reduction of one or more of the adverse side effects by the compound used according to the invention is more than 10% reduction; or alternatively more than 20% reduction; or alternatively more than 30% reduction; or alternatively more than 40% reduction; or alternatively more than 50% reduction; or alternatively more than 60% reduction; or alternatively more than 70% reduction; or alternatively more than 80% reduction; or alternatively more than 90% reduction; or alternatively more than 99% reduction; or alternatively complete reduction of the adverse side effect. In some embodiments, the above recited reduction in the one or more of the adverse side effects is as compared to the adverse side effects of other β-adrenergic agonists. In some embodiments, the above recited reduction in the one or more of the adverse side effects is as compared to the adverse side effects of terbutaline.

Typically, the β-adrenergic agonists suffer from a short half life or low bioavailability. In some embodiments, the compounds used according to the invention have a longer half life or higher bioavailability as compared to other β-adrenergic agonists, such as but not limited to, terbutaline.

### 3. Compounds Useful In Practicing The Invention

The compounds that are used in the methods, compositions, and devices of the invention are as follows.

In one aspect, the compound is of formula I:

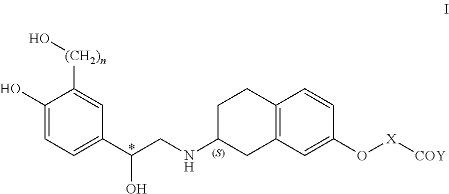

wherein
n is an integer selected from 1 or 2;
X is a C₁-C₆ alkylene group;
Y is —N(R)₂ wherein each R is independently selected from hydrogen or C₁-C₆ alkyl, or two R along with the nitrogen bound thereto join together to form a 3 to 7 membered heterocyclic ring optionally containing an oxygen atom; and
* represents a carbon atom in an R configuration, an S configuration, or a mixture thereof,
a metabolite thereof, a prodrug thereof, or a pharmaceutically acceptable salt of any of the foregoing.

In one aspect, the compound is of formula II:

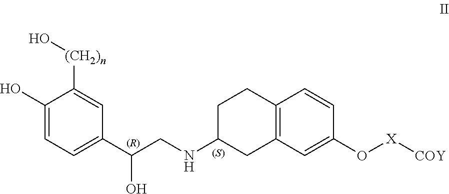

wherein
n is an integer selected from 1 or 2;
X is a C₁-C₆ alkylene group; and
Y is —N(R)₂ wherein each R is independently selected from hydrogen or C₁-C₆ alkyl, or two R along with the nitrogen bound thereto join together to form a 3 to 7 membered heterocyclic ring optionally containing an oxygen atom;
a metabolite thereof, a prodrug thereof, or a pharmaceutically acceptable salt of any of the foregoing.

In one aspect, the compound is of formula III:

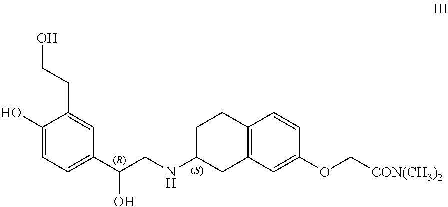

a metabolite thereof, a prodrug thereof, or a pharmaceutically acceptable salt of any of the foregoing.

In one aspect, the metabolite is of formula IV:

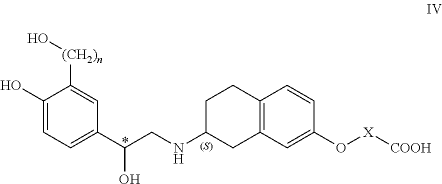

wherein
n is an integer selected from 1 or 2;
X is a C₁-C₆ alkylene group; and
* represents a carbon atom in an R configuration, an S configuration, or a mixture thereof,
or a pharmaceutically acceptable salt thereof.

In some embodiments, the compound is of formula V:

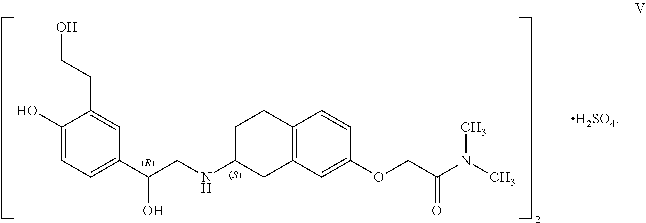

In some embodiments of the above recited aspects, X is a C₁-C₃ alkylene group. In some embodiments of the above recited aspects, X is a —CH₂— group.

In some embodiments of the above recited aspects, Y is —N(R)₂ wherein each R is hydrogen.

In some embodiments of the above recited aspects, Y is —N(R)₂ wherein each R is C₁-C₆ alkyl. In some embodiments of the above recited aspects, Y is —N(R)₂ wherein each R is C₁-C₂ alkyl. In some embodiments of the above recited aspects, Y is —N(R)₂ wherein each R is methyl. In some embodiments of the above recited aspects, Y is —NHR wherein R is C₁-C₂ alkyl.

In some embodiments of the above recited aspects, Y is —N(R)₂ wherein two R along with the nitrogen bound thereto join together to form a 3 to 7 membered heterocyclic ring optionally containing an oxygen atom.

In some embodiments of the above recited aspects, * represents a carbon atom in R configuration. In some embodiments of the above recited aspects, * represents a carbon atom in S configuration. In some embodiments of the above recited aspects, * represents a carbon atom which is a mixture of R and S configuration.

In some embodiments of the above recited aspects, n is 1. In some embodiments of the above recited aspects, n is 2.

The compounds of the invention can exist in unsolvated as well as solvated forms, including hydrated forms. In general, the solvated forms, including hydrated forms and the like are equivalent to the unsolvated forms for purposes of the invention.

In some embodiments, the compound is in a form of a prodrug wherein the prodrug is selected from the group consisting of compounds wherein hydroxyl or amine groups are bonded to a group that, when administered to a subject, cleaves to form a free hydroxyl or amine group, respectively. In some embodiments, the prodrug is selected from the group consisting of acetate, formate, benzoate and phosphate ester derivatives of hydroxyl functional group, and acetyl and benzoyl derivatives of amine functional group.

In some embodiments, the compound or the prodrug thereof is in a form of a pharmaceutically acceptable salt thereof wherein the pharmaceutically acceptable salt thereof is an acid addition salt wherein the acid is selected from the group consisting of hydrochloric, sulfuric, phosphoric, acetic, citric, oxalic, malonic, salicyclic, malic, gluconic, fumaric, succinic, ascorbic, maleic, and methanesulfonic acid. In some embodiments, the pharmaceutically acceptable salt thereof is sulfuric acid.

In some embodiments, the compound is a metabolite thereof where metabolites are as described herein. In some embodiments, the compound is a pharmaceutically acceptable salt of the metabolite of the compound, where pharmaceutically acceptable salts are as described herein.

The compounds used according to the invention can be synthesized using routine synthetic chemistry known to one skilled in the art. For example, the syntheses of the compounds used according to the invention and their experimental data are described in U.S. Pat. No. 6,133,266 and U.S. Pat. No. 6,136,852, which are incorporated herein by reference in their entirety.

### 4. Pharmaceutical Compositions, Devices and Dosages

In one aspect, there is provided a composition comprising a compound used according to the invention and a pharmaceutically acceptable carrier, wherein the composition is suitable for use according to the invention. The compounds used according to the invention can be administered admixed with conventional excipients, such as, pharmaceutically acceptable liquid, semi-liquid or solid organic or inorganic carriers, which do not deleteriously react with the active compound in admixture therewith. Suitable pharmaceutically acceptable carriers include but are not limited to water, salt solutions, alcohols, vegetable oils, polyethylene glycols, gelatin, lactose, amylose, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc.

The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring, and/or aromatic substances and the like which do not deleteriously react with the active compounds.

Various delivery systems are known and can be used to administer the compounds or compositions according to the invention, including, for example, encapsulation in liposomes, microbubbles, emulsions, microparticles, microcapsules and the like. The required dosage can be administered as a single unit or in a sustained release form.

In some embodiments, the composition is administered as a formulation suitable for parenteral routes of administration, such as intravenous, intramuscular, percutaneous, and subcutaneous administration. For parenteral application, particularly suitable are solutions, preferably oily or aqueous solutions, as well as suspensions, emulsions, or implants, including suppositories.

In a related embodiment, the intravenous formulation comprises approximately 0.20 mg to about 20 mg; or alternatively about 0.20 mg to about 10 mg; or alternatively about 0.20 mg to about 5 mg; or alternatively about 0.20 mg to about 3 mg; or alternatively about 0.20 mg to about 2 mg; or alternatively about 0.20 mg to about 1 mg; of the compound used according to the invention in an aqueous delivery system. The aqueous delivery system may comprise about 0.02% to about 0.5% (w/v) of an acetate, phosphate, or citrate buffer. In another aspect, the formulation has a pH of about 3.0 to about 7.0. In a related aspect, the concentration of the compound in the intravenous formulation falls in the range of about 0.15 μmol/mL to about 0.25 μmol/mL.

In some embodiments, the subject is administered an amount of the compound useful according to the invention in the range of about 3 μg/kg patient (or about 200 μg per patient) to about 60 μg/kg patient (or about 4 mg per patient). The dosage may be administered intravenously as a single bolus injection to the subject, or as single bolus injection followed by a constant infusion for up to 24, 36, 48, or 72 hours, or as a constant infusion for up to 24, 36, 48, or 72 hours. The dosage may be administered subcutaneously or intravenously at intervals not less than 4 hours and for up to 24, 36, 48, or 72 hours. In some embodiments, the subject is administered intravenously for 15 minutes at about 40 μg/min and then about 45 minutes at about 13 μg/min. In yet another embodiment, the subjects are those who have been admitted to an emergency room.

In some embodiments, the intravenous formulation is reconstituted from a freeze-dried drug product comprising the compound used according to the invention. In another embodiment, the freeze-dried drug product further comprises carbohydrate and/or polyhydric alcohols. The carbohydrate may be mannose, ribose, trehalose, maltose, inositol, lactose, or the like. The polyhydric alcohols may be sorbitol, mannitol, or the like.

In certain embodiments within the various aspects and embodiments of the present invention, the compound is administered by infusion. In one embodiment, the infusion is performed at a rate of about 3 μg (μgm or μg)/minute to about 60 μg/min; about 6 μg/minute to about 30 μg/minute; about 12 μg/minute to about 15 μg/minute; about 7 μg/minute to about 18 μg/minute; about 9 μg/minute; about 13 μg/minute; and about 16 μg/minute.

The compound is formulated as a liquid formulation for administration in accordance with the various aspects and embodiments of the present invention. In some embodiments, the liquid formulation comprises the compound in an amount of about 3 μg/mL to about 60 μg/mL, about 6 μg/mL to about 30 μg/mL, and about 12 μg/mL to about 30 μg/mL, and about 15 μg/mL to about 20 μg/mL. In another embodiment, the liquid formulation further comprises dextrose. In another embodiment, the liquid formulation is an aqueous formulation. In another embodiment, the liquid formulation is suitable for intravenous injection or infusion.

In the various aspects and embodiments of the present invention, the compound is used as a 2 mg, unit dose, lyophilized drug product. Other unit dose forms in the range of about 0.2 mg to about 20 mg are also contemplated. In one embodiment, the lyophilized drug product further comprises lactose.

In one aspect, the compositions of the invention are delivered topically. Topical administration can involve the use of transdermal administration such as transdermal patches or iontophoresis devices. Dosage forms for topical administration of the compounds and compositions can include creams, sprays, lotions, gels, ointments, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol).

The compositions can also be applied topically using a transdermal system, such as one of an acrylic-based polymer adhesive with a resinous cross-linking agent impregnated with the composition and laminated to an impermeable backing. In some embodiments, the compositions of the present invention are administered in the form of a transdermal patch, such as in the form of a sustained-release transdermal patch. In some embodiments, the compositions of the present invention are administered in a form of a five day transdermal patch.

The transdermal patches of the present invention can include any conventional form such as, for example, adhesive matrix, polymeric matrix, reservoir patch, matrix or monolithic-type laminated structure, and are generally comprised of one or more backing layers, adhesives, penetration enhancers, an optional rate controlling membrane and a release liner which is removed to expose the adhesives prior to application. Polymeric matrix patches also comprise a polymeric-matrix forming material.

In some embodiments, the transdermal patches comprise a therapeutically effective amount of the composition of the invention and optionally an antioxidant. Examples of antioxidants include, but are not limited to, hydralazine compounds, glutathione, vitamin C, vitamin E, cysteine, N-acetyl-cysteine, β-carotene, ubiquinone, ubiquinol-10, tocopherols, coenzyme Q, and the like. Suitable antioxidant enzymes include, but are not limited to, superoxide dismutase, catalase, glutathione peroxidase, and the like. Suitable antioxidants are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996).

In some embodiments, the composition, the transdermal patch, or the delivery device can be a controlled release composition. Non-limiting examples of a suitable biocompatible excipient for applying the compound include a lipophilic carrier or a hydrophilic carrier. Non-limiting examples of a lipophilic carrier include semi-synthetic glycerides of saturated fatty acids. Non-limiting examples of a hydrophilic carrier include polyethylene glycol having an average molecular weight of 6000, polyethylene glycol having an average molecular weight of 1500, polyethylene glycol having an average molecular weight of 400 or mixtures thereof. The biocompatible excipient can also include a muco-adhesive agent such as alginate, pectin, or cellulose derivative. The biocompatible excipient can also include a penetration enhancer such as bile salts, organic solvents, ethoxydiglycol, or interesterified stone oil.

In one embodiment of the invention, the excipient comprises between about 60 to 90% by weight lipophilic carrier, between about 5 to 25% mucoadhesive agent, and between about 5 to 20% penetration enhancer. In another embodiment of the invention, the excipient comprises between about 60 to 90% by weight hydrophilic carrier, between about 5 to 25% muco-adhesive agent, and between about 5 to 20% penetration enhancer. In another embodiment of the invention, the patch or the drug delivery device comprises a standard fragrance free lotion formulation. In another embodiment, the biocompatible excipient can include glycerin, mineral oil, polycarbophil, carbomer 934P, hydrogenated palm oil, glyceride, sodium hydroxide, sorbic acid, and purified water.

In some embodiments, the transdermal patch contains, about 5-5000 mg; or alternatively about 5-4000 mg; or alternatively about 5-3000 mg; or alternatively about 5-2000 mg; or alternatively about 5-1000 mg; or alternatively about 5-500 mg; or alternatively about 5-100 mg; or alternatively about 5-50 mg, of the compound used according to the invention. In some embodiments, the transdermal patch administers a sustained release of the compound used according to the invention over a period of 6 days; or 5 days; or 4 days; or 3 days; or 2 days; or 1 day.

For enteral application, particularly suitable are unit dosage forms, e.g., tablets, dragees or capsules having talc and/or a carbohydrate carrier or binder or the like, the carrier preferably being lactose and/or corn starch and/or potato starch; particulate solids, e.g., granules; and liquids and semi-liquids, e.g., syrups and elixirs or the like, wherein the active compound is protected with differentially degradable coatings, e.g., by microencapsulation, multiple coatings, etc. Suitable for oral administration are, inter alia, tablets, dragees, capsules, pills, granules, suspensions and solutions. Each unit dose, e.g., each tablespoon of liquid or each tablet, or dragee contains, for example, 5-5000 mg of each active agent or the compound used according to the invention.

In some embodiments, the pharmaceutically acceptable carrier is a bioadhesive carrier. In some aspects, the bioadhesive carrier is a cross-linked water-insoluble but water-swellable polycarboxylic acid polymer. The cross-linked polycarboxylic acid polymer formulation, is generally described in U.S. Pat. No. 4,615,697 (hereinafter “the '697 patent”), which is incorporated herein by reference. In general, at least about eighty percent of the monomers of the polymer in such a formulation may contain at least one carboxyl functionality. The cross-linking agent may be present at such an amount as to provide enough bioadhesion to allow the system to remain attached to the target epithelial surfaces for a sufficient time to allow the desired dosing to take place. This preferred level of bioadhesion can be attained when the cross-linking agent is present at about 0.1 to 6.0 weight percent of the polymer, with about 1.0 to 2.0 weight percent being most preferred, as long as the appropriate level of bioadhesion results. Bioadhesion can also be measured by commercially available surface tensiometers utilized to measure adhesive strength.

The polymer formulation can be adjusted to control the release rate of the compounds used according to the invention, by varying the amount of cross-linking agent in the polymer. Suitable cross-linking agents include divinyl glycol, divinylbenzene, N,N-diallylacrylamide, 3,4-dihydroxy-1,5-hexadiene, 2,5-dimethyl-1,5-hexadiene and similar agents. A preferred polymer for use in such a formulation is Polycarbophil, U.S.P., which is commercially available from B.F. Goodrich Speciality Polymers of Cleveland, Ohio under the trade name NOVEON®-M1. The United States Pharmacopeia, 1995 edition, United States Pharmacopeial Convention, Inc., Rockville, Md., at pages 1240-41, indicates that polycarbophil is a polyacrylic acid, cross-linked with divinyl glycol. Polycarbophil is a main ingredient in the vaginal moisturizer Replens®. It has also been used as a base for compositions with other active substances such as progesterone (Crinone®) (see U.S. Pat. No. 5,543,150) and Nonoxynol-9 (Advantage-S) (see U.S. Pat. No. 5,667,492). Other useful bioadhesive polymers that may be used in such a drug delivery system formulation are mentioned in the '697 patent. For example, these include polyacrylic acid polymers cross-linked with, for example, 3,4-dihydroxy-1,5-hexadiene, and polymethacrylic acid polymers cross-linked with, for example, divinyl benzene.

Typically, these polymers may not be used in their salt form, because this would decrease their bioadhesive capability. Such bioadhesive polymers may be prepared by conventional free radical polymerization techniques utilizing initiators such as benzoyl peroxide, azobisisobutyronitrile, and the like. Exemplary preparations of useful bioadhesives are provided in the '697 patent.

The bioadhesive formulation may be in the form of a gel, cream, tablet, pill, capsule, suppository, film, or any other pharmaceutically acceptable form that adheres to the mucosa and does not wash away easily. Different formulations are further described in the '697 patent, which is incorporated herein by reference.

Additionally, the additives taught in the '697 patent may be mixed in with the cross-linked polymer in the formulation for maximum or desired efficacy of the delivery system or for the comfort of the patient. Such additives include, for example, lubricants, plasticizing agents, preservatives, gel formers, tablet formers, pill formers, suppository formers, film formers, cream formers, disintegrating agents, coatings, binders, vehicles, coloring agents, taste and/or odor controlling agents, humectants, viscosity controlling agents, pH-adjusting agents, and similar agents.

The compounds used according to the invention or the other optional drug can be administered as an admixture or as a separate unit dosage form, either simultaneously therewith or at different times during the day from each other. The compound and the optional drug are preferably administered at least once daily (unless administered in a dosage form which delivers the active agents continuously) and more preferable several times daily, e.g., in 2 to 6 divided doses. The typical dose is about 0.5 to 1000 mg of each active agent.

A lower dosage regimen can be initiated and the dosage can be increased until a positive effect is achieved or a higher dosage regimen can initially be employed, e.g., in a crisis situation, and the dosages regulated downward as relief from the symptoms is achieved.

In some embodiments, the method of the invention comprises intravaginal insertion of a device comprising a compound used according to the invention for treatment of dysmenorrhea in a pharmaceutically acceptable, non-toxic carrier. The composition is combined with a suitable delivery device or system which permits the transvaginal delivery of the drug to the uterus through the vaginal mucosa. Examples of the drug delivery system include, but are not limited to, a tampon device, vaginal ring, pessary, tablet, vaginal suppository, vaginal sponge, bioadhesive tablet, bioadhesive microparticle, cream, lotion, foam, ointment, solution and gel. Alternatively, it can be a coating on a suppository wall or a sponge or other absorbent material impregnated with a liquid drug containing solution, lotion, or suspension of bioadhesive particles. Any form of drug delivery system which will effectively deliver the treatment agent to the uterus or the vaginal epithelium is intended to be included within the scope of this invention.

In some embodiments, the device is an absorbent vaginal tampon device having a proximal and a distal end. Located at the distal end is a means for delivery of the compound to the epithelium of the vagina. The device also includes a means for preferentially conveying fluid discharged from the uterus near the proximal end to the tampon and thereby preventing contact of the fluid with the compound. The device also has a means for retrieval of the device, such as a string or tape as used in tampons, vaginal rings and diaphragms. In another embodiment of the invention, the drug delivery device can be a vaginal suppository.

In some embodiments, the compound and an optional drug are in the form of a microsphere for enhancing uptake of the compound and the drug. The microparticles have a diameter of 10-100 pm and can be prepared from starch, gelatin, albumin, collagen, or dextran.

The compound can also be incorporated into creams, lotions, foams, paste, ointments, and gels which can be applied to the vagina using an applicator. Processes for preparing pharmaceuticals in cream, lotion, foam, paste, ointment and gel formats can be found throughout the literature. An example of a suitable system is a standard fragrance free lotion formulation containing glycerol, ceramides, mineral oil, petrolatum, parabens, fragrance and water. Suitable nontoxic pharmaceutically acceptable systems for use in the compositions of the present invention will be apparent to those skilled in the art of pharmaceutical formulations and examples are described in REMINGTON'S Pharmaceutical Sciences, 19th Edition, A. R. Gennaro, ed., 1995. The choice of suitable carriers will depend on the exact nature of the particular dosage form desired, e.g., whether the active ingredient(s) is/are to be formulated into a cream, lotion, foam, ointment, paste, solution, or gel, as well as on the compound.

The excipient can be an inert or inactive substance used in the production of pharmaceutical products or other tablets, including without limitation any substance used as a binder, disintegrant, coating, compression/encapsulation aid, cream or lotion, lubricant, parenteral, sweetener or flavoring, suspending/gelling agent, or wet granulation agent. Binders include, e.g., carbopol, povidone, xanthan gum, etc.; coatings include, e.g., cellulose acetate phthalate, ethylcellulose, gellan gum, maltodextrin, etc.; compression/encapsulation aids include, e.g., calcium carbonate, dextrose, fructose dc, honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams and lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; parenterals include, e.g., mannitol, povidone, etc.; plasticizers include, e.g., dibutyl sebacate, polyvinylacetate phthalate, etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

In certain embodiments within the various aspects and embodiments of the present invention, the compound is administered in an amount of about 2000 μg (or 2 mg), about 1200 μg, about 1000 μg, about 800 μg, about 600 μg, about 450 μg, about 400 μg, about 250 μg, and about 200 μg (or 0.2 mg). In other embodiments, the compound is administered in an amount of about 200 μg to about 2000 μg.

In certain embodiments within the various aspects and embodiments of the present invention, the compound is administered for a period of time up to about 6 days, up to about 5 days, up to about 4 days, up to about 3 days, up to about 2 days, up to about 1 day, up to about 8 hours (h), up to about 2 h, up to about 1 h, up to about 45 min; up to about 30 min, and up to about 15 min. The compound may be administered at various rates of administration, for various periods of time.

Unless otherwise stated all temperatures are in degrees Celsius. Also, in these examples and elsewhere, abbreviations have the following meanings:

| | |
|---|---|
| CaCl₂ = | calcium chloride |
| g = | Gram |
| Kg = | Kilogram |
| KCl = | potassium chloride |
| KH₂PO₄ = | potassium dihydrogen phosphate |
| L = | Liter |
| mg = | Milligram |
| μg = | Microgram |
| MgCl₂ = | magnesium chloride |
| min = | Minute |
| mm = | Millimeter |
| mL = | Milliliter |
| nmol = | Nanomole |
| NaCl = | sodium chloride |
| NaHCO₃ = | sodium bicarbonate |
| Na₂HPO₄ = | sodium phosphate |

The following examples are provided to illustrate select embodiments of the invention as disclosed and claimed herein.

### EXAMPLES

The compound MN-221 in the examples and figures provided herein, refers to the sulfate salt of formula:

### (−)-bis(2-{[(2S)-2-({(2R)-2-hydroxy-2-[4-hydroxy-3-(2-hydroxyethyl)phenyl]ethyl}amino)-1,2,3,4-tetrahydronaphthalen-7-yl]oxy}-N,N-dimethyl-acetamide) monosulfate

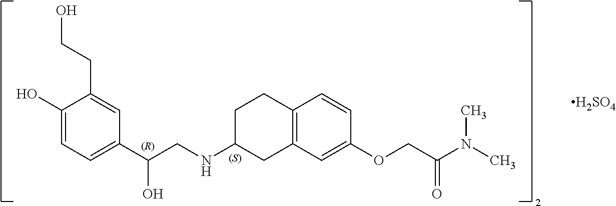

MN-221 can be synthesized according to methods reported in literature. See, e.g., U.S. Pat. No. 6,133,266, which is incorporated herein by reference in its entirety.

The studies provided below use the uterine contraction of the pregnant subjects as models for the study of uterine contraction of female subjects suffering from dysmenorrhea before or during menstruation. Due to the direct effects of MN-221 on smooth muscle contractility, administration of MN-221 proves to be an effective therapy for dysmenorrhea.

The test equipment used in the studies below are: Tension transducer, 45196A, Force displacement transducer 45196A, FD Pick-up SB-1T (force displacement transducer), FD Pick-up TB-611T (force displacement transducer), Amplification unit for conversion 1829 (Strain pressure amplifier), Amplifier case 7747, Amplifier case 7903, Strain pressure amplifier, AP-601G; Amplifier case, RMP-6004; Pen-writing recorder: RECTI-HORIZ 8K10 (Rectigraph), Pen-writing recorder: RECTI-HORIZ 8K20 (Rectigraph), Thermostatic chamber: Thermominder DX-10, Electronic balance PG3001-S, Pan electronic balance MC210S, Electronic balance 1412 MP8, Refrigerated counter for drugs: MPR-1010R, medical freezer, MDF-U332, and Water purification system: Autostil WG-75.

### Example 1

### Effect of MN-221 on Spontaneous Contractions of Uterine Muscle Isolated From Pregnant Rats

This study compares the effect of MN-221 on the spontaneous contractions of the uterine muscle isolated from pregnant rats with that of other β-adrenoceptor agonists.

### Materials

The test substance was MN-221; the control substance was ritodrine hydrochloride ((±)-erythro-1-(p-hydroxyphenyl)-2-[2-(p-hydroxyphenyl)ethylamino]-1-propanol hydrochloride) obtained from Solvay Pharmaceuticals B.V.; and the positive control substance was isoproterenol bitartrate obtained from SIGMA. Other chemicals used in the study were obtained from Nacalai Tesque, Inc.; Otsuka Pharmaceutical Factory, Inc.; and Yoneyama Yakuhin Kogyo Co., Ltd.

Source of rat, Sprague Dawley (SD) strain, 13 weeks old (21 days of pregnancy), was Japan SLC, Inc. A quarantine period of at least 1 week was set. Body weight was measured and general condition observed at the start and end of the quarantine period. Each animal was identified by writing an animal number at the root of the tail with Magic Ink during the quarantine period. The animals were housed in cages as a group of 5 or less. They were allowed to take feed (Rodent diet CE-2 solid food; Clea Japan, Inc.) and drink water (ultraviolet-irradiated tap water of Hotaka-cho) ad libitum. The temperature and humidity of the animal room was kept constant (23° C.±3° C. and 50±10%, respectively). An illumination cycle with a room light being on for 12 hours (from 8:00 am to 8:00 pm) was used.

### Experimental Methods

### 1. Test Groups, Concentrations of Drugs, and Number of Samples

TABLE 1

| | Concentrations of drugs | Number of |
|---|---|---|
| Test groups | (final concentration: mol/L) | samples |
| MN-221 group | 3 × 10⁻¹¹, 1 × 10⁻¹⁰, 3 × 10⁻¹⁰, 1 × 10⁻⁹, | 10 |
| | 3 × 10⁻⁹, 1 × 10⁻⁸, 3 × 10⁻⁸, 1 × 10⁻⁷ | |
| Ritodrine | 1 × 10⁻⁹, 3 × 10⁻⁹, 1 × 10⁻⁸, 3 × 10⁻⁸, | 10 |
| hydrochloride | 1 × 10⁻⁷, 3 × 10⁻⁷, 1 × 10⁻⁶ | |
| group | | |
| Isoproterenol | 1 × 10⁻¹¹, 3 × 10⁻¹¹, 1 × 10⁻¹⁰, 3 × 10⁻¹⁰, | 10 |
| bitartrate | 1 × 10⁻⁹, 3 × 10⁻⁹, 1 × 10⁻⁸ | |
| group | | |

### 2. Preparation of Test, Control, and Positive Control Substances

Each of the substances was weighed and dissolved in distilled water to have a concentration of 1×10⁻² mol/L. Each solution was diluted as required in series (1 to 10) to 1×10⁻⁸ mol/L for MN-221, 1×10⁻⁷ mol/L for ritodrine hydrochloride, and to 1×10⁻⁹ mol/L for isoproterenol bitartrate.

### 3. Preparation of Nutritional Fluid

Locke-Ringer solution: The following substances were weighed and dissolved in distilled water to make 10 L: 90.0 g of NaCl, 4.2 g of KCl, 2.85 g of CaCl₂, 4.25 g of MgCl₂.6H₂O, 5.0 g of glucose, and 5.0 g of NaHCO₃.

### 4. Experimental Operation

The experimental operation of this study was as reported by T. Kawarabayashi et al.¹ After each SD-strain rat on Day 21 of pregnancy were exsanguinated to death, the uterus was isolated to prepare up to 8 myometrial strips (about 4 mm×10 mm) in the direction of the longitudinal muscle, with the adhesion to the placenta being avoided.

Each myometrial strip was suspended in an organ bath containing 10 mL of a Locke-Ringer solution at 37° C. (aerated with 95% O₂+5% CO₂ gas) with a load of about 1.0 g. After the amplitude and frequency of the muscular spontaneous contractions became stable, distilled water was added. Five minutes later, the test, control, or positive control substance was added cumulatively with intervals of 5 minutes. The myometrial contractile force was delivered to a strain pressure amplifier through a force displacement transducer and recorded on a Rectigraph.

### Data Processing Method

### 1. Data Calculation Method

Considering the sum of the uterine contraction during 5 minutes after the addition of distilled water as 100%, the response rate to each concentration of the test, control, or positive control substance was calculated from the sum of the uterine contraction after each was added. Variable points (peaks) with amplitude (tension) of 0.2 g or lower were excluded from analysis. Samples showing any of the following events were not used for the study or excluded from analysis.

### Samples Excluded

1. Sample that did not spontaneously contract 3 times or more in 5 minutes before one of the substances was added.

2. Sample that showed an inhibitory effect by 50% or higher before the second concentration (because the first concentration was set so as to exert almost no inhibitory effect).

3. Sample for which the contraction inhibitory curve as obtained by the cumulative addition of one of the substances crosses the 50% inhibition line 3 times or more (because it makes an EC₅₀ value unclear).

4. Sample that did not show 50% inhibition when each substance was added at its highest concentration (because it was impossible to calculate an EC₅₀ value).

### 2. Statistical Analysis and Processing

Microsoft® Excel 2000 (Microsoft Corp.) was used to sum up and analyze data and prepare tables and figures. Using a concentration-response curve obtained for each sample (X axis: log value of the concentration of the drug added, Y axis: response rate), a negative log value (pD₂) of the concentration that inhibited the uterine contraction by 50% (EC₅₀) was calculated from a straight line that connects the 2 concentrations above and below 50%, and converted to EC₅₀. A mean value and its standard error were calculated for the contraction by each concentration of the test, control, and positive control substance solutions as well as the EC₅₀ and pD₂ values, and presented to 2 decimal places.

SAS system for Windows, Release 8.2, and its associated software, SAS Pre-clinical Package, Version 5.0 (SAS Institute Inc.), were used for statistical analysis. For the inter-group comparison of pD₂ and EC₅₀ values, the variance of each value was examined with Bartlett's test. When the variance was equal, parametric Tukey multiple comparison test was performed. When the variance was not equal, non-parametric Tukey multiple comparison test was performed. In either case, a probability level of less than 5% for both sides was considered to indicate a significant difference. As a result, the results of the parametric Tukey test were used for the pD₂ value, and those of the non-parametric Tukey test for the EC₅₀ value.

### Results

MN-221 inhibited the spontaneous contractions of the uterine muscle isolated from pregnant rats in a concentration-dependent manner (FIG. 1). The EC₅₀ and pD₂ values of the substance were 1.01±0.27 nmol/L and 9.16±0.14, respectively (Table 2). Ritodrine hydrochloride and isoproterenol bitartrate also inhibited the spontaneous contractions in a concentration-dependent manner (FIG. 1). The EC₅₀ (pD₂) value of the ritodrine hydrochloride and isoproterenol bitartrate was 39.81±13.28 nmol/L (7.59±0.13) and 0.42±0.10 nmol/L (9.52±0.13), respectively (Table 2). The inhibitory effect on the spontaneous contractions of the uterine muscle isolated from pregnant rats was observed in the following order: isoproterenol bitartrate≧MN-221>ritodrine hydrochloride.

TABLE 2
Inhibitory effect of β-adrenoceptor agonists on spontaneous
contractions of uterine muscle isolated from pregnant rats

| | Number of | | |
|---|---|---|---|
| Compound | samples | pD₂ | EC₅₀ (nmol/L) |
| MN-221 | 10 | 9.16 ± 0.14* | 1.01 ± 0.27* |
| Ritodrine | 10 | 7.59 ± 0.13^{#} | 39.81 ± 13.28^{#} |
| hydrochloride | | | |
| Isoproterenol | 10 | 9.52 ± 0.13 | 0.42 ± 0.10 |
| bitartrate | | | |

The data represent the mean ± standard error.
*P < 0.05: Indicating a significant difference as compared with ritodrine hydrochloride (Tukey multiple comparison test)
^{#}P < 0.05: Indicating a significant difference as compared with isoproterenol bitartrate (Tukey multiple comparison test).

### REFERENCES

1) Kawarabayashi T, Kobayashi M, Akahane M, Ajisawa Y. Comparison of in vitro and in vivo inhibitory effects of peptide and nonpeptide oxytocin antagonists on radioligand binding and uterine contractility of rats during pregnancy. Am J Obstet Gynecol 1996; 175: 1348-55.

### Example 2

### Interaction of MN-221 and Various β-Adrenoceptor Receptor Antagonists on Spontaneous Contraction of Uterine Muscle from Pregnant Rats

This study demonstrates the action mechanism of MN-221 by examining the interaction between the inhibitory effect of MN-221 on the spontaneous contraction of uterine muscle isolated from pregnant rats and the effect of various β-adrenoceptor antagonists including CGP 20712A¹ (selective β1-adrenoceptor antagonist), ICI 118,551² (selective β2-adrenoceptor antagonist), and SR 59230A³ (selective β3-adrenoceptor antagonist).

### Materials

The test substance was MN-221. Other chemicals used in the study were obtained from Nacalai Tesque, Inc.; SIGMA; Otsuka Pharmaceutical Factory, Inc.; and Yoneyama Yakuhin Kogyo Co., Ltd.

Source of rat, Sprague Dawley (SD) strain, 13 weeks old (21 days of pregnancy), was Japan SLC, Inc. A quarantine period of at least 1 week was set. Body weight of each animal was measured and general condition observed at the start and end of the quarantine period. Each animal was identified by writing an animal number at the root of the tail with Magic Ink during the quarantine period. The animals were housed in cages as a group of 5 or less. They were allowed to take feed (Rodent diet CE-2 solid food; Clea Japan, Inc.) and drink water (ultraviolet-irradiated tap water of Hotaka-cho) ad libitum. The temperature and humidity of the animal room was kept constant (23° C.±3° C. and 50±10%, respectively). An illumination cycle with a room light being on for 12 hours (from 8:00 am to 8:00 pm) was used.

### Experimental Methods

### 1. Test Groups, Concentrations of Drugs, and Number of Samples

TABLE 1

| | Treated | Treated concentration | Number of |
|---|---|---|---|
| Test group | substance | (final concentration) | samples |
| control | Distilled water | — | 10 |
| CGP -9.0 | CGP 20712A | 1 × 10⁻⁹ mol/L | 10 |
| CGP -8.5 | CGP 20712A | 3 × 10⁻⁹ mol/L | 10 |
| CGP -8.0 | CGP 20712A | 1 × 10⁻⁸ mol/L | 10 |
| ICI -8.5 | ICI 118,551 | 3 × 10⁻⁹ mol/L | 10 |
| ICI -8.0 | ICI 118,551 | 1 × 10⁻⁸ mol/L | 10 |
| ICI -7.5 | ICI 118,551 | 3 × 10⁻⁸ mol/L | 10 |
| SR -8.5 | SR 59230A | 3 × 10⁻⁹ mol/L | 10 |
| SR -8.0 | SR 59230A | 1 × 10⁻⁸ mol/L | 10 |
| SR -7.5 | SR 59230A | 3 × 10⁻⁸ mol/L | 12 |

### 2. Preparation of Test Substance and Concentration

An appropriate quantity of MN-221 was weighed and dissolved in distilled water to prepare a solution at 1×10⁻² mol/L, which was diluted with distilled water in series (at a ratio of 1 to 10) to prepare solutions up to 1×10⁻⁸ mol/L as required. Then, MN-221 was cumulatively added in the following concentration ranges.

TABLE 2

| | Concentration of MN-221 (final |
|---|---|
| Test group | concentration: mol/L) |
| control | 3 × 10⁻¹¹, 1 × 10⁻¹⁰, 3 × 10⁻¹⁰, 1 × 10⁻⁹, |
| | 3 × 10⁻⁹, 1 × 10⁻⁸, 3 × 10⁻⁸ |
| CGP | 3 × 10⁻¹¹, 1 × 10⁻¹⁰, 3 × 10⁻¹⁰, 1 × 10⁻⁹, |
| (representing | 3 × 10⁻⁹, 1 × 10⁻⁸, 3 × 10⁻⁸ |
| all CGP groups) | |
| ICI -8.5 | 1 × 10⁻¹⁰, 3 × 10⁻¹⁰, 1 × 10⁻⁹, 3 × 10⁻⁹, |
| | 1 × 10⁻⁸, 3 × 10⁻⁸, 1 × 10⁻⁷, |
| ICI -8.0 | 3 × 10⁻¹⁰, 1 × 10⁻⁹, 3 × 10⁻⁹, 1 × 10⁻⁸, |
| | 3 × 10⁻⁸, 1 × 10⁻⁷, 3 × 10⁻⁷ |
| ICI -7.5 | 1 × 10⁻⁹, 3 × 10⁻⁹, 1 × 10⁻⁸, 3 × 10⁻⁸, |
| | 1 × 10⁻⁷, 3 × 10⁻⁷, 1 × 10⁻⁶ |
| SR | 3 × 10⁻¹¹, 1 × 10⁻¹⁰, 3 × 10⁻¹⁰, 1 × 10⁻⁹, |
| (representing | 3 × 10⁻⁹, 1 × 10⁻⁸, 3 × 10⁻⁸ |
| all SR groups) | |

### 3. Preparation of Nutritional and Other Solutions

Locke-Ringer solution: The following substances were weighed and dissolved in distilled water to make 10 L: 90.0 g of NaCl, 4.2 g of KCl, 2.85 g of CaCl₂, 4.25 g of MgCl₂.6H2O, 5.0 g of glucose, and 5.0 g of NaHCO₃.

CGP 20712A solution: CGP 20712A was weighed and dissolved in distilled water to prepare a solution at 1×10⁻³ mol/L. This solution was used as a stock solution and divided into several portions for cryopreservation. An appropriate portion was diluted with distilled water in series (at a ratio of 1 to 10) to 1×10⁻⁷ mol/L on the experimental day.

ICI 118,551 solution: ICI 118,551 was weighed and dissolved in distilled water to prepare a solution at 1×10⁻³ mol/L. This solution was used as a stock solution and divided into several portions for cryopreservation. An appropriate portion was diluted with distilled water in series (at a ratio of 1 to 10) to 1×10⁻⁶ mol/L on the experimental day.

SR 59230A solution: SR 59230A was weighed and dissolved in DMSO to prepare a solution at 1×10⁻³ mol/L. This solution was used as a stock solution and divided into several portions for cryopreservation. An appropriate portion was diluted with distilled water in series (at a ratio of 1 to 10) to 1×10⁻⁶ mol/L on the experimental day.

### 4. Experimental Operation

The experimental operation of this study was as reported by T. Kawarabayashi et al.⁴. After each SD-strain rat on Day 21 of pregnancy was exsanguinated to death, the uterus was isolated to prepare 8 myometrial samples (about 4 mm×10 mm) in the direction of the longitudinal muscle, with the adhesion to the placenta being avoided. Each myometrial sample was suspended with a load of about 1.0 g in an organ bath containing 10 mL of the Locke-Ringer solution at 37° C. (aerated with 95% O₂+5% CO₂ gas). After the suspended samples became stable for the amplitude and frequency of spontaneous contraction, each antagonist or distilled water was added to the bath to pretreat them for about 15 minutes. Subsequently, a solution of MN-221 (in a concentration range of 3×10⁻¹¹ to 1×10⁻⁶ mol/L as described above) was cumulatively added at intervals of 5 minutes. The contractile force of each sample was outputted through a force displacement transducer to a strain pressure amplifier and recorded with a rectigraph.

### Data Processing

### 1. Data Calculation Method

A response rate to each concentration of MN-221 was calculated as a ratio of the sum of uterine contraction for 5 minutes after it was added to that for 5 minutes before it was added: the sum of uterine contraction before it was added was considered as 100%. Any variable point (peak) with amplitude (tension) of 0.2 g or lower was excluded from analysis. Samples meeting any of the following criteria were rejected or removed from analysis.

### Samples Excluded

1. Sample that did not spontaneously contract at least 3 times in 5 minutes before MN-221 was added.

2. Sample that showed an inhibitory effect of 50% or higher before the third concentration of MN-221 was added (because the drug was added with a starting concentration at which it had almost no inhibitory effect).

3. Sample for which the contraction inhibitory curve as obtained by the cumulative addition of MN-221 crosses the 50% inhibition line 3 times or more (because it makes an EC₅₀ value unclear).

4. Sample not inhibited by at least 50% even when MN-221 was added at its highest concentration (because it was impossible to calculate an EC₅₀ value).

### 2. Statistical Analysis and Processing

Microsoft® Excel 2000 (Microsoft Corp.) was used for summing up or calculating data and preparing figures and tables. Using the concentration response curve prepared for each sample (X axis: logarithmic value of added concentration of MN-221, Y axis: response rate), a negative logarithmic value (pD₂) of the concentration at which uterine contraction was inhibited by 50% (EC₅₀) was calculated from the straight line connecting the response rate at the nearest 2 concentrations above and below 50%, and then converted to EC₅₀ (unit: mol/L). This EC₅₀ value was used to calculate the concentration ratio (CR) of EC₅₀ when an antagonist was added (for each sample) to that when no antagonist was added (mean value of the whole control group) and the logarithmic value of CR-1 (log [CR-1]).

Then, the added concentration of the antagonist was plotted on the X axis (logarithmic value) and log(CR-1) on the Y axis (Schild plot) to calculate the value of the X-axis intercept (pA₂) and slope of the line using linear approximation (Schild regression⁵). The slope was statistically compared with a slope of 1 (paired t-test; a probability level of less than 5% was considered significant). However, it was decided that no Schild regression was performed when the concentration response curve did not clearly shift to the right with the addition of the antagonist. As a result, no Schild regression was performed for the CGP and SR treatment groups because of no evident shift of the concentration response curve to the right. The mean and standard error were calculated for each data point obtained and indicated to two decimal places (or in 3 significant digit).

### Results and Discussion

MN-221 concentration-dependently inhibited the spontaneous contraction of the uterine muscle isolated from pregnant rats (control in figures), with an EC₅₀ (pD₂) value of 0.843±0.221 nmol/L (9.17±0.09) (Table 3). CGP 20712A, a selective β₁-adrenoceptor antagonist, had no evident antagonistic effect on the effect of MN-221 at a concentration of up to 1×10⁻⁸ mol/L (FIG. 2). Similarly, SR 59230A, a selective β₃-adrenoceptor antagonist, had no evident antagonistic effect on the effect of MN-221 at a concentration of up to 3×10⁻⁸ mol/L (FIG. 4). In contrast, ICI 118,551, a selective β₂-adrenoceptor antagonist, had a concentration-dependent, antagonistic effect on the inhibitory effect of MN-221 on uterine contraction (FIG. 3A). The results of the Schild regression showed that the antagonistic effect of ICI 118,551 had a pA₂ value of 9.30±0.11 and a slope of 0.87±0.23, which was not significantly different from a slope of 1 (FIG. 3B).

TABLE 3
Effect of various β-adrenoceptor antagonists on
inhibitory effect of MN-221 on spontaneous contraction
of uterine muscle isolated from pregnant rat

| | Number of | | |
|---|---|---|---|
| Test group | samples | pD₂ | EC₅₀ (nmol/L) |
| Control group (treated | 10 | 9.17 ± 0.09 | 0.843 ± 0.221 |
| with distilled water) | | | |
| CGP 20712A 1 × 10⁻⁹ mol/L | 10 | 8.94 ± 0.06 | 1.25 ± 0.148 |
| treatment group | | | |
| CGP 20712A 3 × 10⁻⁹ mol/L | 10 | 9.16 ± 0.14 | 1.05 ± 0.278 |
| treatment group | | | |
| CGP 20712A 1 × 10⁻⁸ mol/L | 10 | 9.16 ± 0.11 | 0.868 ± 0.150 |
| treatment group | | | |
| ICI 118,551 3 × 10⁻⁹ mol/L | 10 | 8.53 ± 0.15 | 4.60 ± 1.26 |
| treatment group | | | |
| ICI 118,551 1 × 10⁻⁸ mol/L | 10 | 7.78 ± 0.12 | 23.1 ± 6.39 |
| treatment group | | | |
| ICI 118,551 3 × 10⁻⁸ mol/L | 10 | 7.54 ± 0.19 | 50.9 ± 12.7 |
| treatment group | | | |
| SR 59230A 3 × 10⁻⁹ mol/L | 10 | 9.02 ± 0.10 | 1.24 ± 0.338 |
| treatment group | | | |
| SR 59230A 1 × 10⁻⁸ mol/L | 10 | 9.35 ± 0.19 | 1.54 ± 1.08 |
| treatment group | | | |
| SR 59230A 3 × 10⁻⁸ mol/L | 12 | 8.97 ± 0.15 | 1.79 ± 0.499 |
| treatment group | | | |

Each data indicates a mean ± standard error.

Since only ICI 118,551 had a competitive antagonistic effect on the inhibitory effect of MN-221 on uterine contraction, it is contemplated that the inhibitory effect of MN-221 on the spontaneous contraction of the uterine muscle isolated from pregnant rats may be a response via β₂-adrenoceptors.

### REFERENCES

1) Dooley D J, Bittiger H, Reymann N C. CGP 20712A: a useful tool for quantitating beta 1- and beta 2-adrenoceptors. Eur J Pharmacol 1986; 130:137-9.
2) Bilski A J, Halliday S E, Fitzgerald J D, Wale J L. The pharmacyology of a beta 2-selective adrenoceptor antagonist (ICI 118,551). J Cardiovasc Pharmacol 1983; 5: 430-7.
3) Manara L, Badone D, Baroni M, Boccardi G, Cecchi R, Croci T, et al. Functional identification of rat atypical beta-adrenoceptors by the first beta 3-selective antagonists, aryloxypropanolaminotetralins. Br J Pharmacol 1996; 117: 435-42.
4) Kawarabayashi T, Kobayashi M, Akahane M, Ajisawa Y. Comparison of in vitro and in vivo inhibitory effects of peptide and nonpeptide oxytocin antagonists on radioligand binding and uterine contractility of rats during pregnancy. Am J Obstet Gynecol 1996; 175: 1348-55.
5) Arunlakshana O, Schild H O. Some quantitative uses of drug antagonists. Br J Pharmacol 1959; 14: 48-58.

### Example 3

### Effect of MN-221 on Drug-Induced Contraction of Uterine Muscle Isolated From Pregnant Rat

This study demonstrates the effect of MN-221 on prostaglandin (PG) F_{2α}- and oxytocin-induced contractions of uterine muscle isolated from pregnant rats with that of other β-adrenoceptor agonists.

### Materials

The test substance was MN-221; the control substance was ritodrine hydrochloride ((±)-erythro-1-(p-hydroxyphenyl)-2-[2-(p-hydroxyphenyl)ethylamino]-1-propanol hydrochloride) obtained from Solvay Pharmaceuticals B.V.; and the positive control substance was isoproterenol bitartrate obtained from SIGMA. Other chemicals used in the study were obtained from Nacalai Tesque, Inc.; SIGMA; Teikoku Hormone MFG; Ono Pharmaceutical Co., Ltd.; Otsuka Pharmaceutical Factory, Inc.; and Yoneyama Yakuhin Kogyo Co., Ltd.

Source of rat, Sprague Dawley (SD) strain, 13 weeks old (21 days of pregnancy), was Japan SLC, Inc. A quarantine period of at least 3 days was set. Body weight was measured and general condition observed at the start and end of the quarantine period. Each animal was identified by writing an animal number at the root of the tail with Magic Ink during the quarantine period. The animals were housed in cages as a group of 5 or less. They were allowed to take feed (Rodent diet CE-2 solid food; Clea Japan, Inc.) and drink water (ultraviolet-irradiated tap water of Hotaka-cho) ad libitum. The temperature and humidity of the animal room was kept constant (23° C.±3° C. and 50±10%, respectively). An illumination cycle with a room light being on for 12 hours (from 8:00 am to 8:00 pm) was used.

### Experimental Methods

### 1. Test Groups, Concentrations, and Number of Samples

### 1.1 PG F_{2α}-Induced Contraction

TABLE 1

| | Concentrations of drugs | Number of |
|---|---|---|
| Test groups | (final concentration: mol/L) | samples |
| MN-221 group | 1 × 10⁻⁹, 3 × 10⁻⁹, 1 × 10⁻⁸, 3 × 10⁻⁸, | 10 |
| | 1 × 10⁻⁷, 3 × 10⁻⁷, 1 × 10⁻⁶ | |
| Ritodrine | 1 × 10⁻⁸, 3 × 10⁻⁸, 1 × 10⁻⁷, 3 × 10⁻⁷, | 10 |
| hydrochloride | 1 × 10⁻⁶, 3 × 10⁻⁶, 1 × 10⁻⁵ | |
| group | | |
| Isoproterenol | 1 × 10⁻¹⁰, 3 × 10⁻¹⁰, 1 × 10⁻⁹, 3 × 10⁻⁹, | 10 |
| bitartrate | 1 × 10⁻⁸, 3 × 10⁻⁸, 1 × 10⁻⁷ | |
| group | | |

### 1.2 Oxytocin-Induced Contraction

TABLE 2

| | Concentrations of drugs | Number of |
|---|---|---|
| Test groups | (final concentration: mol/L) | samples |
| MN-221 group | 1 × 10⁻¹⁰, 3 × 10⁻¹⁰, 1 × 10⁻⁹, 3 × 10⁻⁹, | 10 |
| | 1 × 10⁻⁸, 3 × 10⁻⁸, 1 × 10⁻⁷ | |
| Ritodrine | 1 × 10⁻⁹, 3 × 10⁻⁹, 1 × 10⁻⁸, 3 × 10⁻⁸, | 10 |
| hydrochloride | 1 × 10⁻⁷, 3 × 10⁻⁷, 1 × 10⁻⁶ | |
| group | | |
| Isoproterenol | 1 × 10⁻¹¹, 3 × 10⁻¹¹, 1 × 10⁻¹⁰, 3 × 10⁻¹⁰, | 10 |
| bitartrate | 1 × 10⁻⁹, 3 × 10⁻⁹, 1 × 10⁻⁸ | |
| group | | |

### 2. Preparation of Test, Control, and Positive Control Substance Solutions

Each of the substances was weighed and dissolved in distilled water to have a concentration of 1×10⁻² mol/L. Each solution was diluted as required in series (1 to 10) to 1×10⁻⁸ mol/L for MN-221, 1×10⁻⁷ mol/L for ritodrine hydrochloride, and to 1×10⁻⁹ mol/L for isoproterenol bitartrate.

### 3. Preparation of Nutritional Fluid and Other Solutions

Modified Locke-Ringer solution: The following substances were weighed and dissolved in distilled water to make 10 L: 88.0 g of NaCl, 4.0 g of KCl, 0.4 g of CaCl₂, 0.38 g of MgCl₂.6H₂O, 0.2 g of KH₂PO₄, 2.02 g of Na₂HPO₄.12H₂O, 5.0 g of glucose, and 4.0 g of NaHCO₃.

PG F_{2α} solution: Prostarmon®-F Injection 1000 (containing 2000 μg of PG F_{2α} in a 2 mL-ampoule) was diluted with distilled water to prepare a 500 μg/mL solution, as required.

Oxytocin solution: Five units of Atonin®-O (containing 5 units of oxytocin in a 1 mL-ampoule) was diluted with distilled water to prepare a 100 mU/mL solution, as required.

Forskolin solution: An appropriate amount of forskolin was weighed and dissolved in DMSO to prepare a 1×10⁻² mol/L solution, which was stored at room temperature in the shade before use.

### 4. Experimental Operation

After SD-strain rats on Day 17 of pregnancy were exsanguinated to death, the uterus was isolated to prepare up to 8 myometrial strips (about 4 mm×10 mm) per animal in the direction of the longitudinal muscle while avoiding the adhesion to the placenta. Each strip was suspended in an organ bath containing 10 mL of a modified Locke-Ringer solution at 26° C. (aerated with 95% O₂+5% CO₂ gas) with a load of about 1.0 g. After the suspended sample became stable for static tension, 5 μg/mL of PG F_{2α} or 1 mU/mL of oxytocin was added to induce contraction. After the sample was allowed to stand for at least 30 minutes to confirm stable rhythmic contraction for frequency and amplitude, the test, control, or positive control substance solution was added cumulatively at intervals of 5 minutes. After the treatment was completed, 1×10⁻⁵ mol/L of forskolin was added to obtain maximal relaxation. The contractile force of the uterine sample was delivered via a force displacement transducer to a strain pressure amplifier and recorded on a Rectigraph.

### Data Processing Method

### 1. Data Calculation Method

Considering the sum of the uterine contraction for 5 minutes before treatment as 100%, the response rate to each concentration of the test, control, or positive control substance solution was calculated from the sum of uterine contraction after treatment for both PG F_{2α} and oxytocin induced contraction. Then, the response rates obtained were used to prepare a concentration-response curve for each sample. The maximum relaxation obtained by adding forskolin was considered as a baseline. Variable points (peaks) with amplitude (tension) of 0. g or lower were excluded from analysis. Samples showing any of the following events were not used for the study or excluded from analysis.

### Samples Excluded

1. Sample that did not produce PGF_{2α} or oxytocin-induced contraction at least 3 times in 5 minutes before test substance treatment.

2. Sample that showed an inhibitory effect by 50% or higher before the second concentration (because the first concentration was set so as to exert almost no inhibitory effect).

3. Sample for which the contraction inhibitory curve as obtained by the cumulative treatment crosses the 50% inhibition line 3 times or more (because no clear EC₅₀ value can be obtained).

4. Sample that did not inhibit the contraction by at least 50% when each solution was added at its highest concentration (because it was impossible to calculate an EC₅₀ value for such a sample).

### 2. Statistical Analysis and Processing

Microsoft® Excel 2000 (Microsoft Corp.) was used to sum up and calculate data and prepare tables and figures. Using a concentration-response curve prepared for each sample (X axis: log value of the concentration of the substance added, Y axis: response rate), a negative log value (pEC₅₀) of the concentration that inhibited the uterine contraction by 50% (EC₅₀) was calculated from a straight line connecting the 2 concentrations just above and below 50%, and then converted to EC₅₀ (unit: mol/L). A mean value and its standard error were calculated for the contraction by each concentration of the test, control, and positive control substance solutions as well as their pEC₅₀ and EC₅₀ values: the mean and standard error of the pEC₅₀ value were expressed to 2 decimal places, and those of the EC₅₀ value as 3 effective digits.

SAS system for Windows, Release 8.2 (SAS Institute Inc.), and its associated software, SAS Pre-clinical Package, Version 5.0 (SAS Institute Japan Inc.), were used for statistical analysis. For inter-group comparison, variance was examined with Bartlett's test. When the variance was equal, parametric Tukey multiple comparison test was performed. When the variance was not equal, non-parametric Tukey multiple comparison test was performed. In either case, a probability level of less than 5% for both sides was considered to indicate a significant difference. As a result, the results of the parametric Tukey test were used for pEC₅₀ value of PG F_{2α}-induced contraction, and those of the non-parametric Tukey test for EC₅₀ value of PG F_{2α}-induced contraction and for EC₅₀ and pEC₅₀ values of oxytocin-induced contraction.

### Results and Discussion

MN-221 inhibited the PG F_{2α}-induced contraction of the uterine muscle isolated from pregnant rats in a concentration-dependent manner (FIG. 5), with EC₅₀ and pEC₅₀ values of 66.4±19.3 nmol/L and 7.29±0.10, respectively (Table 3). Ritodrine hydrochloride and isoproterenol tartrate also inhibited the PG F_{2α}-induced contraction of the uterine muscle isolated from pregnant rats in a concentration-dependent manner (FIG. 5), with an EC₅₀ (pEC₅₀) value of 3430±720 nmol/L (5.58±0.11) and 5.10±0.633 nmol/L (8.32±0.05), respectively (Table 3). The potency of inhibitory effect of MN-221 on the PG F_{2α}-induced uterine contraction was significantly different from that of both ritodrine hydrochloride and isoproterenol bitartrate.

TABLE 3
Inhibitory effects of β-adrenoceptor agonists on PG F_{2α}-induced
contraction of uterine muscle isolated from pregnant rats

| Compounds | pEC₅₀ | EC₅₀ (nmol/L) |
|---|---|---|
| MN-221 group | 7.29 ± 0.10*,^{#} | 66.4 ± 19.37*,^{#} |
| Ritodrine | 5.58 ± 0.11^{#} | 3430 ± 720^{#} |
| hydrochloride | | |
| group | | |
| Isoproterenol | 8.32 ± 0.05 | 5.10 ± 0.633 |
| bitartrate | | |
| group | | |

Uterine contraction was induced with the addition of 5 μg/mL of PG F_{2α}.
The data represent the mean ± standard error of 10 samples.
*P < 0.05: Indicating a significant difference from ritodrine hydrochloride (Tukey multiple comparison test)
^{#}P < 0.05: Indicating a significant difference from isoproterenol bitartrate (Tukey multiple comparison test)

MN-221 inhibited the oxytocin-induced contraction of the uterine muscle isolated from pregnant rats in a concentration-dependent manner (FIG. 6), with EC₅₀ and pEC₅₀ values of 2.25±0.440 nmol/L and 8.73±0.09, respectively (Table 4). Ritodrine hydrochloride and isoproterenol bitartrate also inhibited the oxytocin-induced contraction of the uterine muscle isolated from pregnant rats in a concentration-dependent manner, with an EC₅₀ (pEC₅₀) value of 133±16.8 nmol/L (6.92±0.07) and 0.556±0.0412 nmol/L (9.27±0.03), respectively (Table 4). The potency of inhibitory effect of MN-221 on the oxytocin-induced uterine contraction was significantly different from that of ritodrine hydrochloride, but not from that of isoproterenol bitartrate.

TABLE 4
Inhibitory effects of β-adrenoceptor agonists on oxytocin-
induced contraction of uterine muscle isolated from pregnant rats

| Compounds | pEC₅₀ | EC₅₀ (nmol/L) |
|---|---|---|
| MN-221 group | 8.73 ± 0.09* | 2.25 ± 0.440* |
| Ritodrine | 6.92 ± 0.07^{#} | 133 ± 16.8^{#} |
| hydrochloride | | |
| group | | |
| Isoproterenol | 9.27 ± 0.03 | 0.556 ± 0.0412 |
| bitartrate | | |
| group | | |

Uterine contraction was induced with the addition of 1 mU/mL of oxytocin.
The data represent the mean ± standard error of 10 samples.
*P < 0.05: Indicating a significant difference from ritodrine hydrochloride (Tukey multiple comparison test)
^{#}P < 0.05: Indicating a significant difference from isoproterenol bitartrate (Tukey multiple comparison test)

The highest inhibitory effect on the PG F_{2α}-induced contraction of uterine muscle isolated from pregnant rats was observed with isoproterenol bitartrate, followed by MN-221 and then ritodrine hydrochloride. The highest inhibitory effect on the oxytocin-induced contraction of uterine muscle isolated from pregnant rats was observed with isoproterenol bitartrate, followed by MN-221 and then ritodrine hydrochloride, with no significant difference between the former 2 substances.

### Example 4

### Effects of MN-221 on Uterine Activity of Anesthetized Pregnant Rats

This study demonstrates a comparison of an effect of MN-221 with other β2-adrenoceptor agonists on uterine motility of anesthetized pregnant rats, increases in heart rate of dam and mean blood pressure of dam.

Source of rat, Sprague Dawley (SD) strain, 13 weeks old (21 days of pregnancy), was Japan SLC, Inc. The test substances, MN-221; ritodrine hydrochloride; meluadrine tartrate (HSR-81); and terbutaline sulfate (Sigma), were weighed and dissolved in physiological saline, respectively. Further dilution was performed using physiological saline considering administration dose concentrations.

MN-221 was dosed at 0.1, 0.3, 1.0, 3.0, and 10.0 μg/kg/min.

Ritodrine hydrochloride was dosed at 3.0, 10.0, 30.0, 100.0, and 300.0 μg/kg/min.

Meluadrine tartrate (HSR-81) was dosed at 0.3, 1.0, 3.0, 10.0, and 30.0 μg/kg/min.

Terbutaline sulfate was dosed at 0.3, 1.0, 3.0, 10.0, and 30.0 μg/kg/min.

Rats were anesthetized with urethane, and experiments were conducted based on balloon method. Uterine activity and mean blood pressure of dam were led to a pressure amplifier via a pressure transducer. As for heart rate, pulse waves were led to tachometer. Recti-graphs were used for recording. The test substance, control substance, or positive control substance was administered intravenously and cumulatively every 15 minutes, while doses were increased gradually.

MN-221 and other β2-adrenoceptor stimulants inhibited uterine motility dose-dependently (FIGS. 7 and 8A, Table 1).

TABLE 1
Effects of various β2-adrenoceptor stimulants on uterine motility
of anesthetized pregnant rats: comparisons of ED₃₀ values

| Drug | ED₃₀ values* |
|---|---|
| MN-221 | 0.13 |
| Ritodrine | 51.45 |
| HSR-81 | 0.56 |
| Terbutaline | 0.72 |

*μg/kg/min

The potency of MN-221 was approximately 4 times that of HSR-81, approximately 400 times that of ritodrine and approximately 5.5 times that of terbutaline. All β2-adrenoceptor stimulants used in the experiments, dose-dependently, increased heart rate of dam and decreased mean blood pressure of dam (FIGS. 8B and 8C). However, the effect of MN-221 to increase heart rate was significantly weaker than that of any of other agents and the decrease in mean blood pressure of dam by MN-221 also was significantly small. Therefore, this study demonstrates that MN-221 results in negligible or no adverse side effects in the subject as compared to other β-adrenoceptor stimulants such as terbutaline, ritodrine or HSR-81.

The study also demonstrates that MN-221 at dose that sufficiently inhibits uterine activity has weak actions on heart rate and mean blood pressure of dam, showing that the agent is superior in organ selectivity to other β-adrenoceptor stimulants.

### Example 5

### Effects of MN-221 on Oxytocin-Induced Uterine Contractions, the Cardiovascular System, and General Metabolism of Pregnant Sheep and their Fetuses

This study demonstrates the effects of MN-221 on oxytocin-induced uterine contractions, the cardiovascular system, and general metabolism of pregnant sheep and their fetuses.

The source of sheep, Suffolk strain, 74-88 kg body weight, 118-127 days of pregnancy, was Sankyo Labo Service Co.

MN-221 (0.001, 0.003, 0.01, 0.03, 0.1 and 0.3 μg/kg/min) was weighed and dissolved in physiological saline. At 123-125 days of pregnancy, sheep were infused with oxytocin (1.0 mU/kg/min) to induce uterine contractions. One hour later, MN-221 was infused for 3 consecutive hours beginning at a dose of 0.001 μg/kg/min for 30 min and increasing stepwise every 30 min to 0.3 μg/kg/min in the MN-221 group (N=4). The control received saline instead (N=4).

MN-221 suppressed oxytocin-induced uterine contractions more than 90% at doses over 0.03 μg/kg/min (FIGS. 9 & 10). Significant differences between the two groups were found for the following parameters: maternal heart rate, diastolic blood pressure, mean blood pressure, base excess, blood K+, blood lactate, plasma glucose, plasma insulin, plasma non-esterified fatty acid levels, and fetal plasma glucose and plasma insulin levels (FIGS. 11, 12, 13, 14 & 15). MN-221 significantly inhibited oxytocin-induced uterine contractions at doses over 0.03 μg/kg/min and showed reduced cardiovascular and metabolic side effects.

### Example 6

### Effects of MN-221 on Spontaneous Contractions of Isolated Uterine Muscles of Pregnant Rabbits

This study demonstrates the effects of MN-221 on spontaneous contractions of isolated uterine muscles of pregnant rabbits as compared to other β-adrenoceptor agonists. Source of rabbit, New Zealand White strain, 24 weeks old (29 days of pregnancy), was Kitayama Labes Co. Ltd. The test substances, MN-221; ritodrine hydrochloride; meluadrine tartrate (HSR-81); isoproterenol tartrate (Sigma); and terbutaline sulfate (Sigma), were weighed and dissolved in distilled water, respectively. Further dilution was performed using distilled water considering administration dose concentrations.

MN-221 was dosed at 10⁻¹⁰, 3×10⁻¹⁰, 10⁻⁹, 3×10⁻⁹, 10⁻⁸, 3×10⁻⁸, 10⁻⁷, 3×10⁻⁷, 10⁻⁶ mol/L.

Ritodrine hydrochloride was dosed at 10⁻⁹, 3×10⁻⁹, 10⁻⁸, 3×10⁻⁸, 10⁻⁷, 3×10⁻⁷, 10⁻⁶, 3×10⁻⁶, 10⁻⁵ mol/L.

Meluadrine tartrate (HSR-81) was dosed at 10⁻¹⁰, 3×10⁻¹⁰, 10⁻⁹, 3×10⁻⁹, 10⁻⁸, 3×10⁻⁸, 10⁻⁷, 3×10⁻⁷, 10⁻⁶ mol/L.

Isoproterenol tartrate was dosed at 10⁻¹⁰, 3×10⁻¹⁰, 10⁻⁹, 3×10⁻⁹, 10⁻⁸, 3×10⁻⁸, 10⁻⁷, 3×10⁻⁷, 10⁻⁶ mol/L.

Terbutaline sulfate was dosed at 10⁻⁹, 3×10⁻⁹, 10⁻⁸, 3×10⁻⁸, 10⁻⁷, 3×10⁻⁷, 10⁻⁶, 3×10⁻⁶, 10⁻⁵ mol/L.

Uterine muscles of pregnant rabbits were isolated, and experiments were conducted based on organ-bath method. After specimens of uterine muscles were suspended and spontaneous contractions was stabilized, the test substance, control substance or positive control substance was administered every 10 min, while doses were increased gradually. Efficacy of drugs was evaluated by comparing the sum of uterine contraction for 10 min before and after administration of drugs, defining the former as 100%.

MN-221 and all other drugs used in the test demonstrated inhibitory effect against oxytocin-induced contractions of isolated uterine muscles. Inhibitory effect of MN-221 against spontaneous contractions of uterine muscles was clearly stronger than that of HSR-81, ritodrine and terbutaline (FIG. 16 & Table 1). It is contemplated that MN-221 inhibited spontaneous contractions of isolated uterine muscles of pregnant rabbits through β2-adrenoceptor.

TABLE 1
pD₂ values for the inhibitory effects of MN-221 and other
β2-adrenocepter agonists on spontaneous contractions
of uterine muscles isolated from pregnant rabbits

| Drug | N | pD₂ values |
|---|---|---|
| MN-221 | 8 | 8.72 ± 0.16 |
| Ritodrine hydrochloride | 8 | 6.87 ± 0.12 |
| HSR-81 | 7 | 8.21 ± 0.26 |
| Isoproterenol tartrate | 8 | 8.69 ± 0.17 |
| Terbutaline sulfate | 7 | 7.05 ± 0.24 |

### Example 7

### Effects of MN-221 on Oxytocin-Induced Contractions of Isolated Uterine Muscles of Pregnant Rabbits

This study demonstrates the effects of MN-221 on oxytocin-induced contractions of isolated uterine muscles of pregnant rabbits as compared to other β-adrenoceptor agonists. Source of rabbit, New Zealand White strain, 24 weeks old (29 days of pregnancy), was Kitayama Labes Co. Ltd. The test substances, MN-221; ritodrine hydrochloride; meluadrine tartrate (HSR-81); isoproterenol tartrate (Sigma); and terbutaline sulfate (Sigma), were weighed and dissolved in distilled water, respectively. Further dilution was performed using distilled water considering administration dose concentrations.

MN-221 was dosed at 10⁻¹⁰, 3×10⁻¹⁰, 10⁻⁹, 3×10⁻⁹, 10⁻⁸, 3×10⁻⁸, 10⁻⁷, 3×10⁻⁷, 10⁻⁶ mol/L.

Ritodrine hydrochloride was dosed at 10⁻⁹, 3×10⁻⁹, 10⁻⁸, 3×10⁻⁸, 10⁻⁷, 3×10⁻⁷, 10⁻⁶, 3×10⁻⁶, 10⁻⁵ mol/L.

Meluadrine tartrate (HSR-81) was dosed at 10⁻¹⁰, 3×10⁻¹⁰, 10⁻⁹, 3×10⁻⁹, 10⁻⁸, 3×10⁻⁸, 10⁻⁷, 3×10⁻⁷, 10⁻⁶ mol/L.

Isoproterenol tartrate was dosed at 10⁻¹⁰, 3×10⁻¹⁰, 10⁻⁹, 3×10⁻⁹, 10⁻⁸, 3×10⁻⁸, 10⁻⁷, 3×10⁻⁷, 10⁻⁶ mol/L.

Terbutaline sulfate was dosed at 10⁻⁹, 3×10⁻⁹, 10⁻⁸, 3×10⁻⁸, 10⁻⁷, 3×10⁻⁷, 10⁻⁶, 3×10⁻⁶, 10⁻⁵ mol/L.

Uterine muscles of pregnant rabbits were isolated, and experiments were conducted based on organ-bath method. After specimens of uterine muscles were suspended and oxytocin (1 mU/mL)-induced contractions was stabilized, the test substance, control substance or positive control substance was administered every 10 min, while doses were increased gradually. Efficacy of drugs was evaluated by comparing the sum of uterine contraction for 10 min before and after administration of drugs, defining the former as 100%.

MN-221 and all other drugs used in the test demonstrated inhibitory effect against oxytocin-induced contractions of isolated uterine muscles. Inhibitory effect of MN-221 against oxytocin-induced contractions of uterine muscles was clearly stronger than that of HSR-81, ritodrine and terbutaline (FIG. 17 & Table 1). It is contemplated that MN-221 inhibited oxytocin-induced contractions of isolated uterine muscles of pregnant rabbits through β-adrenoceptor.

TABLE 1
pD₂ values for the inhibitory effects of MN-221 and other
β2-adrenocepter agonists on oxytocin-induced contractions
of uterine muscles isolated from pregnant rabbits

| Drug | N | pD₂ values |
|---|---|---|
| MN-221 | 8 | 8.60 ± 0.09 |
| Ritodrine hydrochloride | 8 | 7.11 ± 0.09 |
| HSR-81 | 8 | 8.12 ± 0.21 |
| Isoproterenol tartrate | 8 | 8.53 ± 0.21 |
| Terbutaline sulfate | 8 | 7.39 ± 0.19 |

### Example 8

### Effects of MN-221 on Myometrial Activity, Uterine Blood Flow, and Lower Abdominal Pain in Women with Primary Dysmenorrhea

Myometrial activity and uterine blood flow is recorded in a group of women, aged 16 to 39 years. All have regular cycles of 25 to 32 days. They suffer regularly from menstrual pain. All are so disabled by the condition that they have to abstain from work for one to three days a month, even if they use non-narcotic analgesics. At least during the first menstrual day, all have a continuous lower abdominal pain, which varies in intensity; most of them also complain of symptoms such as nausea and vomiting. The symptoms during the recordings conform to those experienced during previous menstruations.

In the menstrual cycle preceding the investigation, all women have biphasic basal body temperature recordings, and plasma oestradiol and progesterone concentrations which are higher in the middle of the luteal phase than on the first menstrual day. Recordings are made; each begin within 24 hours of the onset of menstruation lasting for more than three hours.

Myometrial activity is recorded as changes in intrauterine pressure by a microtransducer catheter. The transducer is connected to an amplifier and a potentiometer recorder. Uterine blood flow is recorded by a technique based on measuring thermodilution from a heated thermistor to blood flow in the surrounding tissue. The thermistor is placed in contact with the endometrium of the fundus and, consequently, the recordings mainly reflect the blood flow at that site. The thermistor and the pressure transducer are inserted through the cervical canal into the uterus. The receptors are kept in position by the rigidity of the transducer catheter and by use of sterile paste around the catheters in the vagina.

The patients are asked to report all symptoms during recordings, including changes in character and intensity of the menstrual pain. MN-221 is given as a single bolus intravenous injection of 0.30 mg, 0.60 mg, or 0.90 mg. Before and after the administration of MN-221, pulse rate and arterial blood pressure (measured by auscultation) are registered at 5-minute intervals.

### Recordings Before Administration of MN-221

The maximum intensity of the intrauterine pressure in the different women varies between 200 and 350 mm Hg. The duration of the contractions, namely the time when the intrauterine pressure is higher than the basal tone, generally varies between 1.5 and 3 minutes, and contractions occur with a frequency of about 20 to 40 per hour.

During the contractions, double or multiple peaks of intrauterine pressure are seen. The first of the peaks is usually the highest. The basal tone, which is given by the microtransducer, varies. It is generally between 50 mm Hg-75 mm Hg. During well demarcated contractions: the local uterine blood flow invariably decreases. However, the minimum flow usually occurs somewhat after the maximum intrauterine pressure. The decrease in blood flow is most pronounced during contractions of high amplitude and long duration, and at times of frequent contractions without periods of relaxation between them.

Recordings after Administration of MN-221

The response to MN-221 is qualitatively the same in all the women with respect to myometrial activity, local uterine blood flow, and pain. MN-221 decreases the myometrial activity where the uterine contractions are either inhibited by the drug or appear with a lower frequency and amplitude. Furthermore, there are well defined periods of relaxation between the contractions. The local uterine blood flow generally increases after the drug is given.

Patients report pain relief within one minute after injection, or after infusion of MN-221. When the maximum effect on the blood flow is reached and when the myometrial activity is reduced or abolished, the patients are completely free from pain.

The effect of MN-221 lasts for few hours; the pain then gradually returns. The myometrial contractions and the associated variations in local uterine blood flow resume their original pattern. In all subjects, MN-221 causes substantially no increase in heart rate, blood pressure, palpitations, tremors, and/or flushes.

### Example 9

### Effects of Transdermal Administration of MN-221 for Alleviation of Severe Pain in Women with Primary Dysmenorrhea

This study investigates an effect of transdermal administration of MN-221 on lower abdominal pain in women with severe primary dysmenorrhea.

The study is conducted in women, aged 15-39 years. Some of them are nulliparous and some are parous. All have regular 25-32 day cycles. They have all suffered from severe dysmenorrhea for more than one year. All the women are incapacitated for 1-2 days every month. During the first menstrual day they have continuous low abdominal pain of varying severity and in some women it is accompanied by nausea and vomiting.

All the women are informed and give their consent before the trial is started. The study is performed as a double-blind cross-over trial in which the patients are given, in random order, MN-221 transdermal patches during one menstrual period and placebo patches of identical appearance during the next period. The patients return to the hospital after the end of each menstrual flow and their dysmenorrheic symptoms are assessed. The therapeutic effect is also assessed and graded as none, weak, and moderate, over to good and very good. All women in the study have signs of ovulation—mid-cycle temperature rise—in the menstrual cycle preceding the trial. The MN-221 or the placebo is given as a five day transdermal patch during menstrual pains. MN-221 is administered as a dose in a range of about 0.02 mg/kg to 1.5 mg/Kg. The volume of the menstrual blood loss is calculated from the hemoglobin content of all the sanitary towels used by the patient and delivered to the department.

MN-221 gives a positive response with relief in women as compared to placebo. The difference is statistically significant. The MN-221 transdermal patch provides relief to menstruating women throughout their menstrual cycle. In all subjects, MN-221 causes substantially no increase in heart rate, blood pressure, palpitations, tremors, and/or flushes.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method for a prevention and/or treatment of dysmenorrhea or amelioration of a symptom thereof in a subject, comprising administering to said subject an effective amount of a compound of formula I: 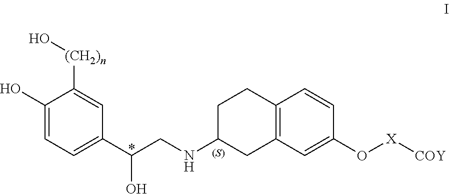
wherein
n is an integer selected from 1 or 2;
X is a C₁-C₆ alkylene group;
Y is —N(R)₂ wherein each R is independently selected from hydrogen or C₁-C₆ alkyl, or two R along with the nitrogen bound thereto join together to form a 3 to 7 membered heterocyclic ring optionally containing an oxygen atom; and
* represents a carbon atom in an R configuration, an S configuration, or a mixture thereof,
a metabolite thereof, a prodrug thereof, or a pharmaceutically acceptable salt of any of the foregoing.

2. The method of claim 1, wherein the compound is of formula II: 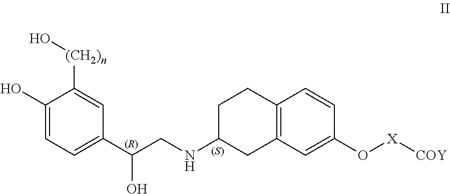
wherein
n is an integer selected from 1 or 2;
X is a C₁-C₆ alkylene group; and
Y is —N(R)₂ wherein each R is independently selected from hydrogen or C₁-C₆ alkyl, or two R along with the nitrogen bound thereto join together to form a 3 to 7 membered heterocyclic ring optionally containing an oxygen atom;
a metabolite thereof, a prodrug thereof, or a pharmaceutically acceptable salt of any of the foregoing.

3. The method of claim 1, wherein the compound is of formula III: 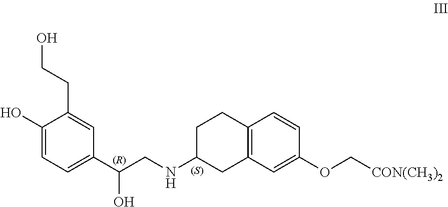
a metabolite thereof, a prodrug thereof, or a pharmaceutically acceptable salt of any of the foregoing.

4. The method of claim 1, wherein the metabolite is of formula IV: 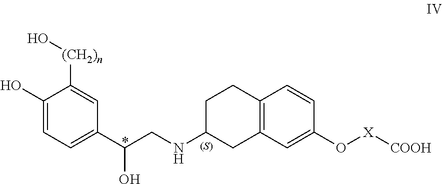
wherein
n is an integer selected from 1 or 2;
X is a C₁-C₆ alkylene group; and
* represents a carbon atom in an R configuration, an S configuration, or a mixture thereof,
or a pharmaceutically acceptable salt thereof.

5. A method for a prevention and/or treatment of dysmenorrhea or amelioration of a symptom thereof in a subject, comprising administering to said subject an effective amount of a compound of formula V:

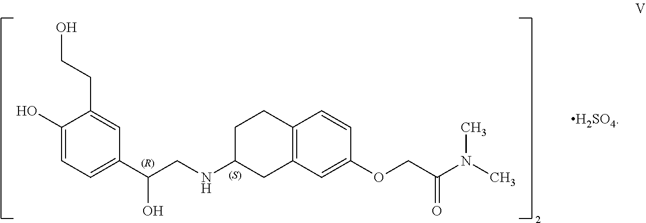

6. The method of claim 5, wherein the subject is a female mammal.

7. The method of claim 5, further comprising administering to said subject a drug selected from the group consisting of non-steroidal anti-inflammatory drugs, anti-prostaglandins, prostaglandin inhibitors, COX-2 inhibitors, local anesthetics, calcium channel blockers, potassium channel blockers, leukotriene blocking agents, smooth muscle inhibitors, vasodilators, and drugs capable of inhibiting dyskinetic muscle contraction.

8. The method of claim 7, wherein the drug is administered concomitantly or sequentially to said composition.

9. The method of claim 5, wherein the administration is oral.

10. The method of claim 9, wherein the compound is in a form of a tablet, capsule, gel or solution.

11. The method of claim 5, wherein the administration is parenteral selected from intravenous, intramuscular, intraarterial, percutaneous, or subcutaneous.

12. The method of claim 5, wherein the administration is transdermal.

13. The method of claim 5, wherein the prodrug is selected from the group consisting of compounds wherein hydroxyl or amine groups are bonded to a group that, when administered to a subject, cleaves to form a free hydroxyl or amine group, respectively.

14. The method of claim 5, wherein the prodrug is selected from the group consisting of acetate, formate, benzoate and phosphate ester derivatives of hydroxyl functional group, and acetyl and benzoyl derivatives of amine functional group.

15. The method of claim 5, wherein the pharmaceutically acceptable salt is an acid addition salt wherein the acid is selected from the group consisting of hydrochloric, sulfuric, phosphoric, acetic, citric, oxalic, malonic, salicyclic, malic, gluconic, fumaric, succinic, ascorbic, maleic, and methanesulfonic acid.

16. The method of claim 5, wherein the compound is in a composition comprising a pharmaceutically acceptable carrier.

17. The method of claim 5, wherein the compound is administered in a dose of about 0.0001 to about 500 mg per kilogram of body weight per day.

18. The method of claim 5, wherein the administration of the compound reduces the incidence of one or more adverse side effects in the subject.

19. The method of claim 18, wherein the number of incidences of the one or more of adverse side effects in the subject is reduced compared to the number of such incidences, which would have been observed in the subject with the administration of terbutaline, ritodrine, or meluadrine.

20. The method of claim 18, wherein the one or more adverse side effects are selected from palpitations; peripheral tremors; high heart rate; low blood pressure; pulmonary edema; hypoglycemia; aggravation of preexisting diabetes; aggravation of preexisting keto acidosis; tremors; nervousness; increased heart rate; dizziness; headaches; drowsiness; vomiting; nausea; sweating; muscle cramps; and electrocardiogram (ECG) changes.

21. The method of claim 18, wherein the number of incidences of increased heart rate, decrease in mean blood pressure, or both in the subject is reduced compared to the number of such incidences, which would have been observed in the subject with the administration of terbutaline.
